(19) United States
(12) Patent Application Publication
(10) Pub. No.: US 2016/0206714 A1
(43) Pub. Date: Jul. 21, 2016

(54) Diagnostic Test and Treatment/Prevention of Alzheimer's Disease

(71) Applicant: VITRUVIAN BIOMEDICAL, INC., Addison, TX (US)

(72) Inventors: Arthur P. Bollon, Dallas, TX (US); BaoXi Qu, Ashburn, VA (US)

(21) Appl. No.: 14/911,381

(22) PCT Filed: May 15, 2015
(86) PCT No.: PCT/US2015/031011

§ 371 Date: Feb. 10, 2016

Related U.S. Application Data
(60) Provisional application No. 61/994,644, filed on May 16, 2014.

(51) Int. Cl. A61K 39/00; C12Q 1/68; G01N 33/68

(52) U.S. Cl. CPC A61K 39/0007; G01N 33/6896; C12Q 1/6883; G01N 2800/2821; G01N 2333/47; G01N 2800/52; A61K 2039/57; A61K 2039/53; C12Q 2600/158

(57) ABSTRACT

The present invention includes a method for diagnosis and treatment and prevention of Alzheimer's Disease comprising obtaining a biological sample from a subject suspected of having Alzheimer's Disease; determining the level of expression of HSP 27, wherein a statistically significant increase in HSP27 protein expression in the sample as compared to a sample from a non-Alzheimer's patient is indicative that the subject has Alzheimer's Disease; and modifying the treatment of the subject as a result of the detection of Alzheimer's Disease by providing the subject with standard therapy or a single vector expressing an Aβ42 trimer peptide and optionally the addition of an Aβ42 peptide, which elicits an immune reaction against the Aβ42 peptide, thereby preventing the accumulation of Aβ42 peptide and therefore preventing or treating Alzheimer's Disease.

### TECHNICAL FIELD OF THE INVENTION

The present invention relates in general to the field of Alzheimer's Disease, and more particularly, to a diagnostic method and compositions and methods for treatment and prevention of Alzheimer's Disease.

### BACKGROUND OF THE INVENTION

Without limiting the scope of the invention, its background is described in connection with Alzheimer's Disease.

U.S. Pat. No. 7,479,550, issued to U.S. Pat. No. 7,479,550, issued to Rosenberg, et al., is entitled “Amyloid β gene vaccines.” This invention includes compositions and methods for genetic vaccination with amyloid beta (Aβ) protein. The vaccine is said to provide effective treatment for neurodegenerative disease such as Alzheimer's disease. Vaccination methods are can be used to induce a Th2 type immune response directed to Aβ. This immune response is said to substantially reduce Aβ concentration and Aβ plaque size in an Alzheimer's model system. One challenge with the use of this system is the need to use two separate vectors known as the Gal4/UAS system. The Gal4/UAS system was effective in inducing an immune response against the Amyloid ABeta-42 peptide in a transgenic mouse model, resulting in inhibition of Amyloid ABeta-42 accumulation. However, the two-vector system, also called a binary vector system, uses two plasmid vectors, that impose a greater production burden, sterility issues and suboptimal use with patients. A single plasmid vaccine would be ideal for both production and application in the clinic.

U.S. Pat. No. 4,816,388, issued to Sipe, et al., is entitled “Human prealbumin and related methods and products”. Briefly, this patent is said to teach, in addition to recombinant human prealbumin, the use of human prealbumin cDNA in the diagnosis by hybridization methodologies of medical conditions with which variant forms of prealbumin are associated, namely, diagnosing Type I familial amyloid polyneuropathies by a restriction endonuclease assay with an enzyme which recognizes the nucleotide base sequence 5′-ATGCAT-3′.

United States Patent Application Publication No. 2014/0031245, filed by Khan, et al., is entitled Alzheimer's Disease-Specific Alterations of the ERK1/ERK2 Phosphorylation Ratio-Alzheimer's Disease-Specific Molecular Biomarkers (ADSMB). Briefly, this application is said to teach methods of diagnosing Alzheimer's Disease as well as to methods of confirming the presence or absence of Alzheimer's Disease in a subject. The present invention identifies a lead compound useful for the treatment of Alzheimer's Disease by contacting non-Alzheimer's cells with an amyloid beta peptide, stimulating the cells with a protein kinase C activator, contacting the cells with a test compound, and determining the value of an Alzheimer's Disease-specific molecular biomarker. The application is also said to teach methods of diagnosing Alzheimer's Disease in a subject by detecting alterations in the ratio of specific phosphorylated MAP kinase proteins in cells after stimulation with a protein kinase C activator.

United States Patent Application Publication No. 2012/0192294, filed by Heneka, et al., is entitled “Inhibitors of the Nitration of Amyloid Beta Peptides and Their Uses in the Diagnosis and Treatment of Alzheimer's Disease.” Briefly, this application is said to teach a method for identifying an inhibitor of the aggregation of amyloid-β peptide (Aβ), comprising the steps of a) contacting at least one Aβ-peptide and/or the nitrated forms thereof with at least one candidate inhibitor that potentially specifically binds to a region in said Aβ-peptide capable of being nitrated, and b) detecting said inhibitor specifically binding to said region in said Aβ-peptide through detecting a lack of or a reduced aggregation of said at least one Aβ-peptide. The present invention is further directed at improved methods for treating neuronal degradation and particularly Alzheimer's disease, based on said inhibitor. The present invention is further directed at methods for diagnosing the aggregation of Aβ-peptide in the context of neuronal degradation and particularly Alzheimer's disease.

### SUMMARY OF THE INVENTION

The present invention includes a diagnostic test for Alzheimer's Disease based on the protein levels and/or RNA expression levels of the protein Heat Shock Protein 27 (HSP27) in patient samples such as tissue, fluids such as blood or other bodily elements from patients who had or were predisposed to disease such as Alzheimer Disease. The levels of HSP27 would be determined in the patient samples using ELISA, nucleic acid hybridization, Nano-BioSensor technology or other detection systems. The diagnostic test was then used to direct treatment or prevention of Alzheimer's Disease in potential patients and patients with a novel expression vector.

In one embodiment, the present invention includes a method for diagnosis and treatment and prevention of Alzheimer's Disease comprising: obtaining a biological sample from a subject suspected of having Alzheimer's Disease; determining the level of expression of HSP 27, wherein a statistically significant increase in HSP27 protein expression in the sample as compared to a sample from a non-Alzheimer's patient is indicative that the subject has Alzheimer's Disease; and modifying the treatment of the subject as a result of the detection of Alzheimer's Disease by providing the subject with standard therapy or a composition comprising a single DNA vector encoding the Aβ42 trimer peptide, wherein the expressed Aβ42 trimer peptide triggers an immune response to the Aβ42 peptide. In one aspect, the subject is a human. In another aspect, the HSP27 is human HSP27. In another aspect, the composition further comprises an Aβ42 peptide and the composition comprising the DNA vector and the Aβ42 peptide is injected intramuscularly without the need for a gene gun or gold particles. In another aspect, the level of HSP 27 is determined by measuring protein expression, and the method is selected from fluorescence detection, chemiluminescence detection, electrochemiluminescence detection and patterned arrays, antibody binding, fluorescence activated sorting, detectable bead sorting, antibody arrays, microarrays, enzymatic arrays, receptor binding arrays, solid-phase binding arrays, liquid phase binding arrays, fluorescent resonance transfer, or radioactive labeling. In another aspect, the level of expression of HSP27 is determined at the nucleic acid level, and the method is selected from fluorescence detection, chemiluminescence detection, electrochemiluminescence detection and patterned arrays, reverse transcriptase-polymerase chain reaction, detectable bead sorting, microarrays, enzymatic arrays, allele specific primer extension, target specific primer extension, solid-phase binding arrays, liquid phase binding arrays, fluorescent resonance transfer, or radioactive labeling. In another aspect, the level of expression of HSP27 is higher than 85, 90, 95, 100, 110, 115, 120, 125, 130, 145, 150, 275, 300, or 315 ng/ml HSP27 in a blood sample. In another aspect, the level of expression of HSP27 is higher than 105, 130, 145, 150, 275, 300, or 315 ng/ml HSP27 in a blood sample. In another aspect, the expressed Aβ42 trimer peptide triggers a non-inflammatory IgG1 response and not an IgG2a or IgG2b response. In another aspect, the Aβ42 peptide and the DNA vector expressing the Aβ42 trimer peptide are effective to trigger an immune response to the Aβ42 peptide without an adjuvant.

Another embodiment of the present invention include a method to evaluate a candidate drug believed to be useful in treating Alzheimer's Disease, the method comprising: (a) measuring the level of expression of HSP27 from a sample obtained from an Alzheimer's Disease patient; (b) administering a candidate drug to a first subset of the patients, and a placebo to a second subset of the patients, wherein the candidate drug comprises a single DNA vector encoding an Aβ42 trimer peptide; (c) determining if the level of expression of HSP27 or the symptoms of Alzheimer's Disease decreased in the first set of patient as compared to the second subset of patients, wherein a statistically significant decrease is indicative that the candidate drug is useful for treating Alzheimer's Disease. In one aspect, the subject is a human. In another aspect, the drug candidate further comprises an Aβ42 trimer peptide and the DNA vector and the Aβ42 trimer peptide are injected intramuscularly without the need for a gene gun or gold particles. In another aspect, the HSP27 is human HSP27. In another aspect, the level of HSP 27 is determined by measuring protein expression, and the method is selected from fluorescence detection, chemiluminescence detection, electrochemiluminescence detection and patterned arrays, antibody binding, fluorescence activated sorting, detectable bead sorting, antibody arrays, microarrays, enzymatic arrays, receptor binding arrays, solid-phase binding arrays, liquid phase binding arrays, fluorescent resonance transfer, or radioactive labeling. In another aspect, the level of expression of HSP27 is determined at the nucleic acid level, and the method is selected from fluorescence detection, chemiluminescence detection, electrochemiluminescence detection and patterned arrays, reverse transcriptase-polymerase chain reaction, detectable bead sorting, microarrays, enzymatic arrays, allele specific primer extension, target specific primer extension, solid-phase binding arrays, liquid phase binding arrays, fluorescent resonance transfer, or radioactive labeling. In another aspect, the level of expression of HSP27 is higher than 85, 90, 95, 100, 110, 115, 120, 125, 130, 145, 150, 275, 300, or 315 ng/ml HSP27 in a blood sample. In another aspect, the level of expression of HSP27 is higher than 105, 130, 145, 150, 275, 300, or 315 ng/ml HSP27 in a blood sample. In another aspect, the DNA vector is a single DNA vector.

Yet another embodiment of the present invention includes compositions, methods, pharmaceuticals, methods of making, using and compositions manufactured to treat or prevent Alzheimer's Disease that include a single vector comprising: a single nucleic acid that comprises in the following order a viral gene leader sequence, a Aβ42 trimer sequence, and an endosomal targeting sequence. In one aspect, the viral gene leader sequence is an adenovirus E3 gene leader sequence. In another aspect, the vector further comprises a CMV promoter upstream from the nucleic acid. In another aspect, the vector comprises SEQ ID NO: 1. In another aspect, the wherein the endosomal targeting sequence is, e.g., DXXLL (SEQ ID NO: 2), or can be obtained from the human invariant (II) chain. In another aspect, the vector is PV1-H3. In another aspect, the vector is PV1-H3 is used to treat or prevent Alzheimer's Disease.

Yet another embodiment of the present invention includes a composition for ameliorating the symptoms of Alzheimer's Disease comprising an Aβ42 trimer peptide and a DNA vector encoding the Aβ42 trimer peptide in an amount sufficient to ameliorate the symptoms of Alzheimer's Disease. In one aspect, the composition is provided without an adjuvant. In another aspect, the composition triggers a predominantly Th2 response. In another aspect, the Aβ42 trimer peptide and the DNA vector are injected intramuscularly without the need for a gene gun or gold particles. In one aspect, the DNA vector is a single DNA vector. In another aspect, the peptide comprises SEQ ID NO: 3. In another aspect, the vector comprises SEQ ID NO: 1. In another aspect, the composition consists essentially of the vector of SEQ ID NO: 1 and the peptide of SEQ ID NO: 3.

In yet another embodiment, the present invention includes a composition for ameliorating the symptoms of Alzheimer's Disease comprising both an Aβ42 peptide and a DNA vector that expresses an Aβ42 trimer peptide in an amount sufficient to ameliorate the symptoms of Alzheimer's Disease, wherein the Aβ42 peptide and the DNA vector that expresses the Aβ42 trimer peptide are both injected intramuscularly without the need for a gene gun or gold particles (and a use of the same), wherein the composition triggers an immune response to the Aβ42 peptide. In one aspect, the expressed Aβ42 trimer peptide and the DNA vector are provided without an adjuvant. In another aspect, the DNA vector is a single DNA vector. In another aspect, the composition leads to a predominantly Th2 response. In another aspect, the peptide comprises SEQ ID NO: 3. In another aspect, the vector comprises SEQ ID NO: 1. In another aspect, the composition consists essentially of the vector of SEQ ID NO: 1 and the peptide of SEQ ID NO: 3.

In another embodiment, the present invention includes a method for the treatment or prevention of Alzheimer's Disease comprising injecting a composition that includes both an Aβ42 peptide and a DNA vector that expresses an Aβ42 trimer peptide, wherein the Aβ42 peptide and the DNA vector are adapted for injection intramuscularly without the need for a gene gun or gold particles (and a use of the same), wherein the composition triggers an immune response to the Aβ42 peptide. In another aspect, the injection triggers a non-inflammatory IgG1 response. In another aspect, the Aβ42 peptide and the DNA vector are provided without an adjuvant. In another aspect, the DNA vector is a single DNA vector. In another aspect, the composition leads to a predominantly Th2 response. In another aspect, the Aβ42 peptide comprises SEQ ID NO: 3. In another aspect, the vector comprises SEQ ID NO: 1. In another aspect, the composition consists essentially of the vector of SEQ ID NO: 1 and the peptide of SEQ ID NO: 3.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the DNA binding to gold particles. The optimal ratio of DNA to the gold is 4.5 ug DNA (p4u-Ab42 trimer) with 1 mg gold. In this ratio, about 3.8 ug Ab42 trimer DNA can be bind to 1.5 mg gold per cartridge (Bullet) plus 20% CMVi-Gal4 DNA as additional.

FIG. 2 is a schematic presentation of single plasmid vector for DNA vaccine against Alzheimer's disease. The Amyloid ABeta-42 trimer gene was cloned between a CMV (pCMV) promoter upstream and SV40 PolyA downstream.

FIG. 3 shows that the single plasmid of the present invention is 2× more active than a binary system.

FIG. 4 shows an antibody isotyping of the antibody generated by the single plasmid PV1-H3—which is a non-inflammatory profile.

FIG. 5 is a graph that shows the results from 4 muscle injections (once a week (20 ug) with trimer DNA+10 ug Aβ peptide (or separate injection) and tested the antibodies at 6 weeks. It was found that DNA+Peptide without adjuvant elicit a better immune response.

FIG. 6 is a graph that shows the results from 4 muscle injections 4 times (once a week) muscle injection (20 ug Trimer DNA+10 ug Aβ peptide), the serum was tested for Abeta isotype antibodies at 6 weeks with ELISA method. It was found that DNA+Peptide without adjuvant elicited a better immune response compare to peptide alone. Higher isotype antibodies level achieved in DNA+Peptide, but both group induced Th1 and Th2 reaction with predominantly Th2 (IgG1 and IgG2a) bias.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “Amyloid ABeta-42” taught herein refers to the nucleotides encoding the Amyloid ABeta-42 peptide variant taught herein that is a portion of the entire vector set forth (SEQ ID NO: 1), and that has amino acid sequence SEQ ID NO: 2.

The terms “a sequence essentially as set forth in SEQ ID NO: (#)”, “a sequence similar to”, “nucleotide sequence” and similar terms, with respect to nucleotides, refers to sequences that substantially correspond to any portion of the sequence identified herein as SEQ ID NO: 1. These terms refer to synthetic as well as naturally derived molecules and includes sequences that possess biologically, immunologically, experimentally, or otherwise functionally equivalent activity, for instance with respect to hybridization by nucleic acid segments, or the ability to encode all or portions of Amyloid ABeta-42 or Amyloid ABeta-42 activities. Naturally, these terms are meant to include information in such a sequence as specified by its linear order.

The terms “a sequence essentially as set forth in SEQ ID NO: 2”, “a sequence similar to”, “amino acid sequence” and similar terms, with respect to amino acids, refers to peptides, polypeptides, proteins, fragments, fusions, derivatives and alterations thereof that substantially correspond to the sequences of SEQ ID NO: 2. These terms refer to synthetic as well as naturally derived molecules and includes sequences that possess biologically, immunologically, experimentally, or otherwise functionally equivalent activities, for instance, segments of amino acids which possess immunological activity as an antigenic determinant Naturally, these terms are meant to include information in such a sequence as specified by its linear order.

The term “gene” is used to refer to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “vector” is used in reference to nucleic acid molecules that transfer DNA segment(s) from one cell to another. The vector may be further defined as one designed to propagate the sequences, or as an expression vector that includes a promoter operatively linked to the Amyloid ABeta-42 gene sequence taught herein, or one designed to cause such a promoter to be introduced. The vector may exist in a state independent of the host cell chromosome, or may be integrated into the host cell chromosome.

The term “host cell” refers to cells that have been engineered to contain nucleic acid segments for the Amyloid ABeta-42 gene taught herein, or altered segments, whether archeal, prokaryotic, or eukaryotic. Thus, engineered, or recombinant cells, are distinguishable from naturally occurring cells that do not contain recombinantly introduced genes through the hand of man.

As used herein, the term “endosomal targeting sequence” refers to an amino acid sequence that targets a polypeptide (or portion thereof) that when included in the polypeptide (e.g., fused or conjugated to the polypeptide), increases endosomal localization of the polypeptide. Endosomal targeting signals for directing molecules to endosomes are known in the art and the sequences can be incorporated in expression vectors such that fusion proteins will contain the endosomal targeting signal are produced, see e.g., Sanderson et al. (Proc. Nat'l. Acad. Sci. USA 92:7217-7221, 1995), Wu et al. (Proc. Nat'l. Acad. Sci. USA 92:11671-11675, 1995) and Thomson et al (J. Virol. 72:2246-2252, 1998), which describe endosomal targeting signals (including invariant chain Ii and lysosomal-associated membrane protein LAMP-1) and their use in directing antigens to endosomal and/or lysosomal cellular compartments. Thus, endosomal targeting sequences can include the entire sequence or only a small portion of a targeting sequence such as, e.g., human invariant chain, and can even be included in a pro-polypeptide that is removed one the polypeptide reaches the endosome. One of ordinary skill in the art can readily determine an endosomal targeting portion of a targeting molecule and use well-known molecular biology techniques to make a recombinant fusion protein that include the endosomal targeting sequence. Additional endosomal targeting sequences can be identified by one of ordinary skill in the art and tested for targeting to the HLA class II peptide presentation pathway using no more than routine experimentation.

HSP27. The twenty-seven kiloDalton heat shock protein (Hsp27) belongs to the small heat shock protein family, which are ATP-independent chaperones. The most important function of Hsp27 is based on its ability to bind non-native proteins and inhibit the aggregation of incorrectly folded proteins maintaining them in a refolding-competent state. Additionally, it has anti-apoptotic and antioxidant activities.

Alzheimer's disease (AD) is characterized by pathological lesions such as senile plaques (SP), cerebral amyloid angiopathy (CAA) and neurofibrillary tangles (NFT), predominantly consisting of the incorrectly folded proteins amyloid-β (Aβ) and tau respectively. The extracellular expression of Hsp27 has been observed in classic SP, and in astrocytes associated with both SP and CAA. Amyloid-β (Aβ) and tau proteins found within the pathological lesions induces neuronal loss and cognitive deficits and is believed to be a prominent cause of AD. Although a great amount of work has gone into studying Alzheimer's disease AD, there is currently no accurate or sensitive technique to determine the onset of AD.

The present inventors determined that early in the process of Alzheimer's disease (AD) the dead and dying cells within pathological lesions such as senile plaques (SP), cerebral amyloid angiopathy (CAA) and neurofibrillary tangles (NFT) release their cellular content which makes its way into the systemic circulation. Since Hsp27 makes up a high proportion of the pathological lesions, the inventors developed a simple, sensitive blood test for Hsp27 to determine the early onset of Alzheimer's disease (AD).

Using Heat Shock Protein 27 (HSP27) as a Biomarker.

The present invention includes a diagnostic test for Alzheimer Disease based on the protein levels and/or RNA expression levels of the protein Heat Shock Protein 27 (HSP27) in patient samples such as tissue, fluids such as blood or other bodily elements from patients who had or were predisposed to any disease including Cancer and Alzheimer Disease. The levels of HSP27 are determined in the patient samples using ELISA, nucleic acid hybridization, Nano-BioSensor technology or other detection systems.

HSP27 is a 27,000 dalton member of the Heat Shock Protein (HSP) family. The HSP proteins are ATP-independent chaperones, which work to maintain the integrity of protein structure such as folding of the protein. Perturbations to such structural protein integrity are associated with different disease states. One example is the incorrect folding of Aβ amyloid, which is associated with the early steps involved in Alzheimer's Disease.

A diagnostic test was developed to determine the levels of HSP27 proteins and/or expression levels of the HSP27 gene in blood, tissue or other body elements as an indication of the existence of or prediction of diseases such as Alzheimer's Disease, cancer and other diseases. An ELISA immunology test specific for HSP27 was employed to identify the HSP27 protein levels in patient samples and in samples from individuals which do not have the disease in question and the HSP27 protein levels are compared. If the samples from the patients with the disease show a statistically higher level of the HSP27 (or a lower level depending on the disease), then that could be the basis for a diagnostic test for that particular disease. In addition to comparing the levels of HSP27 proteins in disease (or pre-disease) samples and non-disease samples, the levels of the HSP27 mRNA in disease and non-disease samples can also be determined by hybridization using DNA or other nucleic acid probes. One example of such a methodology to improve diagnostic tests is Nano-BioSensor technology (one example is Guided-Mode Resonance Sensor Technology), which permits detection of the HSP27 protein or mRNA without the need for tags such as radio-isotopes or chemical tags such as Biotin and permit reading the results in real time.

Hsp27 in blood samples is indicative of the early onset of Alzheimer's disease (AD). To test this hypothesis we obtained blood samples from 8 patients recently determined to have early onset Alzheimer's disease (AD) as determined from clinical records and 5 normal age and sex matched subjects. Plasma proteins were recovered from the blood and tested for the concentration of phosphorylated Hsp27 (pHsp27) using the classical sandwich enzyme linked immunosorbant assay (ELISA). Briefly, blood was drawn from patients and added to tubes containing EDTA, centrifuged and the plasma was recovered, aliquoted and stored at −80° C. The total protein content of each aliquot was determined by Bradford analysis using bovine serum albumin as a standard. The samples were then admixed with 1% Lubrol WX for 10 minutes at 4° C. with gentle rocking and pHsp27 content measured by standard sandwich ELISA. Briefly, 96-well microtitre plates (Nunc Immunoplate Maxisorp; Life Technologies) were coated with murine monoclonal anti-human pHsp27 in carbonate buffer, pH 9.5 (2 μg/mL) overnight at 4° C. Plates were then washed with PBS containing 1% Tween-20 (PBS-T) and blocked by incubation with 1% bovine serum albumin in PBS-T. Supernatant was added and bound pHsp27 was detected by the addition of rabbit polyclonal anti-pHsp27 antibody. Bound polyclonal antibody was detected with alkaline phosphatase-conjugated murine monoclonal antibody to rabbit immunoglobulins (Sigma Chemical Co), followed by p-nitrophenyl phosphate substrate (Sigma Chemical Co). The resultant absorbance was measured at 405 nm with a BioRad Benmark Plus plate reader. Standard dose-response curves were generated in parallel with pHsp27 (0 to 20,000 ng/mL; StressGen), and the concentrations of pHsp27 were determined by reference to these standard curves with ASSAYZAP data analysis software (BIOSOFT). The inter-assay variability of the pHsp27 immunoassays was <10%. The results demonstrate that there was a significant increase in pHsp27 in the plasma of 8/8 patients with Alzheimer's disease (AD) as compared to the 5 normal subjects (Table 1).

TABLE 1
Measurement of Hsp27 levels in patients with early
onset Alzheimer's disease (AD).

| Patient number | Mean Hsp72 concentration (ng/ml ± SD)a |
|---|---|
| Control #12-581 | 60 ± 10 |
| Control #12-254 | 85 ± 15 |
| Control #12-542 | 65 ± 13 |
| Control #12-154 | 45 ± 11 |
| Control #12-488 | 82 ± 15 |
| AD #12-658 | 125 ± 12* |
| AD #12-325 | 145 ± 13* |
| AD #12-782 | 225 ± 28* |
| AD #12-489 | 138 ± 15* |
| AD #12-770 | 149 ± 25* |
| AD #12-333 | 308 ± 50* |
| AD #12-411 | 149 ± 15* |
| AD #12-807 | 108 ± 23* |

Data are plasma pHsp27 concentrations from control (normal subjects) and patients with Alzheimer's disease (AD) measured using the classical pHsp27 ELISA as described in detail in the Materials and Methods section. Data is the mean concentration of pHsp27 (ng/ml±SD) and is the sum of three independent experiments performed in quadruplicates. *, p<0.001 vs control (normal subjects).

The results show a mean HSP27 concentration (ng/ml) of 60, 85, 65, 45, 82 in the five non-Alzheimer Disease blood samples and HPS27 concentration (ng/ml) of 125, 145, 225, 138, 149, 308, 149, 108 in the eight Alzheimer Disease blood samples. Hence, there is a statistically significant (p<0.001) increase in the level of HSP27 in the blood of Alzheimer Disease patients compared to non-Alzheimer Disease patient blood. Therefore this is a disclosure that a Diagnostic test based on the levels of HSP27 in blood samples from individuals could indicate the presence of Alzheimer Disease.

A comparable study with a larger number of samples from a larger number of Alzheimer's Disease and non-Alzheimer's Disease individuals. In addition, determination can be made of the HSP42 levels in family members who carry PS1 or PS2 gene defects but have no symptoms of Alzheimer Disease. Individuals who have PS1 or PS2 gene defects have greater than 90% chance of getting Alzheimer Disease starting at an age of approximately 45-50 as opposed to age 65-70 or older, which is common for other forms of the disease. High levels of HSP27 in PS1 or PS2 asymptomatic patients may proceed to develop Alzheimer's Disease, as such, the levels of HSP27 are predictive of Alzheimer's Disease before symptoms; hence a Diagnostic Test showing HSP27 levels can be predictive and could be used to screen the blood of the general population for Alzheimer's Disease.

Once diagnosis is made using the present method, or other methodologies, including standard Alzheimer's Disease clinical testing, the present invention also includes a novel nucleic acid vector with enhanced delivery to target cells, and enhanced expression of functional Aβ42 trimer peptide.

### Construction of the Plasmid.

The present inventors have constructed a Single Plasmid Vector that has the three copies of the Amyloid ABeta-42 gene, cloned between the CMV promoter upstream and SV40 polyA downstream (FIG. 2). Surprisingly, the Single Plasmid Vector PV1-H3, is more active and induces two-fold more antibody against the Amyloid ABeta-42 compared to the Two Vector System. This was surprising because it was assumed that the activity would be about the same as the initial two-vector system. However, the present invention finds the advantage of being a single vector system making manufacturing, regulatory approval, and clinical utility, less complex in addition to the advantage of demonstrating more activity.

FIG. 2 is a schematic presentation of single plasmid vector for DNA vaccine against Alzheimer's disease. The Amyloid ABeta-42 trimer gene was cloned between a CMV (pCMV) promoter upstream and SV40 PolyA downstream.

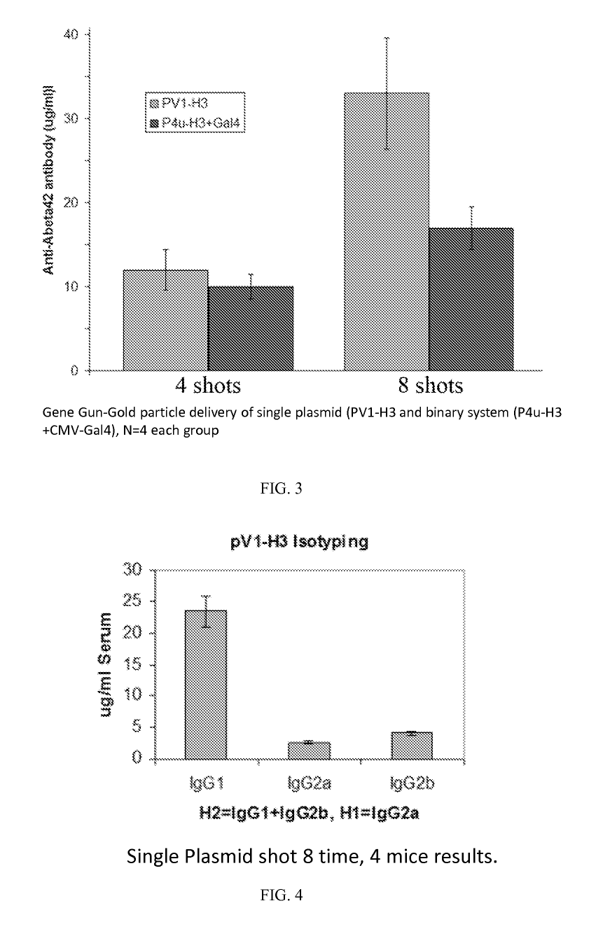

FIG. 3 shows that the single plasmid of the present invention is 2× more active than a binary system. Briefly, the single plasmid PV1-H3 was found to induce about 2× more antibody against Amyloid ABeta-42 when 8 applications are used (33 ug/ml of Anti-Abeta42 antibody by the Single Vector PV1-H3 compared to 16 ug/ml of the Anti-Abeta42 antibody by the Binary System P4u-H3).

FIG. 4 shows an antibody isotyping of the antibody generated by the single plasmid PV1-H3—which is a non-inflammatory profile.

It was also found, surprisingly, that the PV1-H3 Single Plasmid of the present invention generated predominately IgG1 Antibody and minor amounts of IgG2a and IgG2b. The IgG1 antibody is not involved in the inflammatory response. Previous studies using the Amyloid ABeta-42 Peptide itself as a Vaccine, not the gene, induced equal amounts of IgG1 and IgG2a which resulted in an inflammatory response. These results confirm that the PV1-H3 generates predominantly IgG1 which is not inflammatory.

A highly efficient Single Vector, PV1-H3, has been created by the present inventors, which induces two fold higher levels of Antibody against Amyloid ABeta-42 Peptide than the two vector system and the Antibody generated is 90% IGg1 which is characteristic of a non-inflammatory response.

The Aβ42 Trimer Genes were Chemically Synthesized and Cloned into the Immunization Vector System.
1. A set of complementary oligonucleotides of the A.42 DNA sequence were designed using the DNA builder program and custom synthesized (Sigma, St. Louis, Mo.).
2. These oligonucleotides were designed after the respective Aβ42 amino acid sequence using multiple codons for a particular amino acid allowing a more flexible design of the nucleotide sequence to avoid hairpins, primer dimer structures and other inappropriate matches among the sequences which can hinder gene synthesis by polymerase chain reaction (PCR).
3. A total of 32 oligonucleotides (end concentration 250 nM) were mixed for the first PCR reaction to assemble them and built the designed gene sequence (30 cycles: 94° C. for 15 s, 55° C. for 30 s and 72° C. for 45 s; Platinum® Taq DNA Polymerase, Invitrogen, Carlsbad, Calif.).
4. A second PCR was used to amplify the full-length product using a forward and a reverse primer (30 cycles: 94° C. for 15 s, 55° C. for 30 s and 72° C. for 45 s).
5. PCR products from this second run were purified by gel electrophoresis, digested with restriction enzymes (Promega, Madison, Wis.) and cloned into the polycloning site of the plasmid vector (EcoRI/XbaI digestion).
6. Bacteria were transformed with the ligated plasmids and clones were identified by sequence analysis (Applied Biosystem, CA, Sequencing core of UTSW).
7. An adenovirus E3 gene leader sequence and an endosomal targeting sequence were cloned up and down stream of the Aβ42 gene, respectively.
8. For the control immunizations corresponding plasmids were constructed. Plasmid pGal4/UAS-Luc consists of the same binary plasmid system as pGal4/UAS-Aβ42 trimer or monomer but without the E3 leader and endosomal targeting sequence, in which the transcription of the Luc gene is driven by binding of the Gal4 transcription factor. In pCMV-Luc, transcription is driven by a CMV promoter.

### DNA Purification.

All plasmid DNAs were purified using a commercial plasmid maxi kit (Qiagen, Valencia, Calif.). The purity and concentration of DNA were measured by optical density reading at 260/280 nm and gel electrophoresis. Qiagen endotoxin-free DNA purification kit may be needed for electroporation vaccine.

### DNA-Gold Particle Preparations (Advanced Protocol for Clinic Preparation).

1. In a 1.5 ml microfuge tube (Siliconized, Fisher brand #05-541-27), weigh 60 mg gold microcarriers (Degussa Corporation Comgitm # and Batch #33451 60021-05.
2. Wash twice with 100% alcohol, dry in 40° C.
3. Add 270 ug of p4u-Ab42 trimer (routinely 70 ug for mouse vaccine) and 54 ug of pCMVi-ga14, (routinely 14 ug) total DNA 324 ug (routinely 74 ug).
4. Add 100 μl of 0.05 M spermidine.
5. Vortex the gold and spermidine mixture for 10 seconds.
6. While vortexing the mixture at moderate rate speed vortexer, add 100 μl 2.5 M CaCl₂ dropwise to the mixture.
7. Allow the mixture to precipitate on ice for 15 minutes.
8. Spin the microcarrier solution in a microfuge 1 minute (3000 rpm) to pellet the gold.
9. Remove the supernatant and discard.
10. Wash the pellet three times with 1 ml of fresh 100% ethanol each time.
11. After the final ethanol wash, resuspend the pellet in 1.5 ml of the ethanol.
12. The suspension is now ready for tube preparation. Alternatively, the DNA/microcarrier suspensions can be stored for up to 2 months at −20° C. Prior to freezing, tighten the cap securely and put Parafilm® around the cap of the tube. After storage at −20° C., allow the particle suspension to come to room temperature prior to breaking the Parafilm seal.
13. Loading the DNA/Microcarrier Suspension into Gold-Coat Tubing Using the Tubing Prep Station.
14. Allow the microcarriers to settle for 3-5 minutes. Suck out the ethanol.
15. Flow nitrogen in 0.35-0.4 LPM of nitrogen to dry the Gold-Coat tubing.
16. Continue drying the Gold-Coat tubing while turning for 3-5 minutes.
17. Remove the tubing from the tubing support cylinder.
18. Cut into 0.5″ cartridges put into a container.
19. Cap the container tightly, label, wrap with Parafilm, and store at −20° C.
20. The gold particle per cartridge (bullet) is about 1.5 mg gold with about 3.8 ug P4U-Ab42 trimer and 0.96 ug CMVi-Gal4 after freezing for 24 hours and thaw once. The DNA amount per bullet will further be tested after one week and one month storage in −20° C. and P4U-Ab42 trimer should be in about 3.5 ug per bullet.

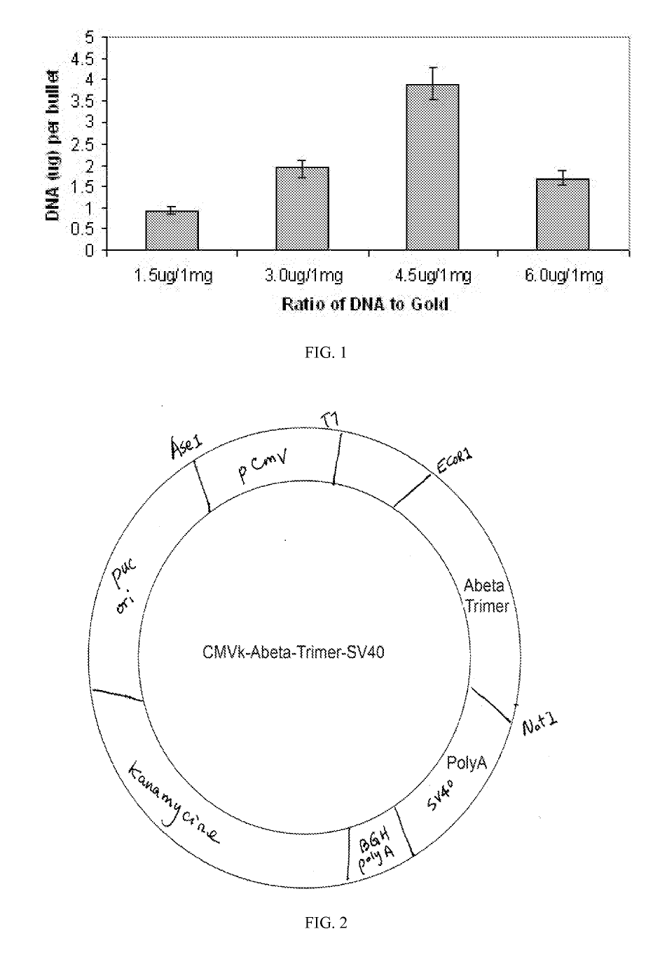

FIG. 1 is a graph that shows the DNA binding to gold particles. The optimal ratio of DNA to the gold is 4.5 ug DNA (p4u-Ab42 trimer) with 1 mg gold. In this ratio, about 3.8 ug Ab42 trimer DNA can be bind to 1.5 mg gold per cartridge (Bullet) plus 20% CMVi-Gal4 DNA as additional.

### Plasmid DNA Sequence.

### P4U-H3 (Abeta Trimer).

Sequence: p4UK-H3 Range: 1 to 4600

```
          >AseI >SnaBI
            |  |
            | 10 |  20 30 40 50
TAGTTATTAATTACGTAGGCTTAACTATGCGGCATCAGAGCAGATTGTAC
ATCAATAATTAATGCATCCGAATTGATACGCCGTAGTCTCGTCTAACATG
                                                        >SbfI
                                                            |
                      >HindIII >PstI
                            |  |
              60 |  70 80|  90 100
TGAGAGTGCACCATAAGCTTGCATGCCTGCAGGTCGAAGCGGAGTACTGT
ACTCTCACGTGGTATTCGAACGTACGGACGTCCAGCTTCGCCTCATGACA
                ___1 TO 527 OF P4U-AB42TRIMERAMP_______> 110
                                  120 130 140 150
CCTCCGAGCGGAGTACTGTCCTCCGAGCGGAGTACTGTCCTTCGAGCGGA
GGAGGCTCGCCTCATGACAGGAGGCTCGCCTCATGACAGGAAGCTCGCCT
________1 TO 527 OF P4U-AB42TRIMERAMP.SEQ_________>
                          >HincII >XhoI
                                |  |
                          >AccI >TliI
                              ||  |
                        >SalI >PaeR7I
                            |||  |
              160 |||170 180 190 |  200
GTACTGTCCTCCGAGTCGACTCTAGAGGGTATATAATGGATCTCGAGATG
CATGACAGGAGGCTCAGCTGAGATCTCCCATATATTACCTAGAGCTCTAC
________1 TO 527 OF P4U-AB42TRIMERAMP.SEQ_________>
                                      >TspMI >XhoI
                                              |  |
                                        >XmaI >TliI
                                              |  |
                    >MluI >BmtI >PaeR7I
                        |  ||  |
            >SacI |  >NheI |>SmaI |  >BglII
                |  |  |  || |  |  |
              210 |  220 || |  230 |  240 250
TACCGAGCTCTTACGCGTGCTAGCCCGGGCTCGAGATCTGGGCGGTAGGC
ATGGCTCGAGAATGCGCACGATCGGGCCCGAGCTCTAGACCCGCCATCCG
________1 TO 527 OF P4U-AB42TRIMERAMP.SEQ_________>
                                                                  >SacI
                                                                      |
              260 270 280 |  290 300
GTGTACGGTGGGAGGTCTATATAAGCAGAGCTCGTTTAGTGAACCGTCAG
CACATGCCACCCTCCAGATATATTCGTCTCGAGCAAATCACTTGGCAGTC
________1 TO 527 OF P4U-AB42TRIMERAMP.SEQ_________>
                                >BmtI
                                    |
                >NheI |
                    |  |
              310|  |  320 330 340 350
ATCACTAGAAGCTAGCTTTATTGCGGTAGTTTATCACAGTTAAATTGCTA
TAGTGATCTTCGATCGAAATAACGCCATCAAATAGTGTCAATTTAACGAT
________1 TO 527 OF P4U-AB42TRIMERAMP.SEQ_________>
                                                                                                  >PstI
                                                                                                      |
              360 370 380 390 |  400
ACGCAGTCAGTGCTTCTGACACAACAGTCTCGAACTTAAGCTGCAGAAGT
TGCGTCAGTCACGAAGACTGTGTTGTCAGAGCTTGAATTCGACGTCTTCA
________1 TO 527 OF P4U-AB42TRIMERAMP.SEQ_________>
              410 420 430 440 450
TGGTCGTGAGGCACTGGGCAGGTAAGTATCAAGGTTACAAGACAGGTTTA
ACCAGCACTCCGTGACCCGTCCATTCATAGTTCCAATGTTCTGTCCAAAT
________1 TO 527 OF P4U-AB42TRIMERAMP.SEQ_________>
              460 470 480 490 500
AGGAGACCAATAGAAACTGGGCTTGTCGAGACAGAGAAGACTCTTGCGTT
TCCTCTGGTTATCTTTGACCCGAACAGCTCTGTCTCTTCTGAGAACGCAA
________1 TO 527 OF P4U-AB42TRIMERAMP.SEQ_________>
              510 520 530 540 550
TCTGATAGGCACCTATTGGTCTTACTGACATCCACTTTGCCTTTCTCTCC
AGACTATCCGTGGATAACCAGAATGACTGTAGGTGAAACGGAAAGAGAGG
________1 TO 527 OF P4U-AB42TRIMERAMP.SEQ_________>
                                                                                              >EcoRI
                                                                                                  |
              560 570 580 590 |  600
ACAGGTGTCCACTCCCAGTTCAATTACAGCTCTTAAGGCTAGAATTCCAC
TGTCCACAGGTGAGGGTCAAGTTAATGTCGAGAATTCCGATCTTAAGGTG
___1 TO 527 OF P4U-AB42TRIMERAMP.SEQ_____>
                                                                                              ___>
                      610 620 630 640 650
GCCGCCACCATGGGCTACATGATCCTGGGCCTCCTGGCCCTGGCGGCCGT
CGGCGGTGGTACCCGATGTACTAGGACCCGGAGGACCGGGACCGCCGGCA
______534 TO 1196 OF P4U-AB42TRIMERAMP.SEQ________>
            >AfeI >MluI
            |  |
                    |660 |  670 680 690 700
GTGCAGCGCTGCCACGCGTGGAGGCGGGAGCGACGCCGAGTTCCGCCACG
CACGTCGCGACGGTGCGCACCTCCGCCCTCGCTGCGGCTCAAGGCGGTGC
______534 TO 1196 OF P4U-AB42TRIMERAMP.SEQ________>
                                      ___61 TO 633 OF AB42-TRIMER >
                                      ___79 TO 723 OF HAB42TRIMER >
>BmgBI
                                                                                                    | 710
                                  720 730 740 750
ACAGCGGCTACGAGGTGCACCACCAGAAGCTGGTGTTCTTCGCCGAGGAC
TGTCGCCGATGCTCCACGTGGTGGTCTTCGACCACAAGAAGCGGCTCCTG
______534 TO 1196 OF P4U-AB42TRIMERAMP.SEQ________>
__________61 TO 633 OF AB42-TRIMER.ENDO___________>
___79 TO 723 OF HAB42TRIMER.E3.ENDO.SEQ [SPLIT____>
                760 770 780 790 800
GTGGGCAGCAACAAGGGCGCCATCATCGGCCTGATGGTGGGCGGCGTGGT
CACCCGTCGTTGTTCCCGCGGTAGTAGCCGGACTACCACCCGCCGCACCA
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
                810 820 830 840 850
GATCGCCGCAGCCTACGATGCGGAATTTCGACATGACAGTGGATATGAAG
CTAGCGGCGTCGGATGCTACGCCTTAAAGCTGTACTGTCACCTATACTTC
______534 TO 1196 OF P4U-AB42TRIMERAMP.SEQ________>
__________61 TO 633 OF AB42-TRIMER.ENDO___________>
___79 TO 723 OF HAB42TRIMER.E3.ENDO.SEQ [SPLIT____>
                860 870 880 890 900
TACATCACCAAAAACTCGTATTTTTCGCGGAAGATGTAGGAAGCAACAAG
ATGTAGTGGTTTTTGAGCATAAAAAGCGCCTTCTACATCCTTCGTTGTTC
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
                910 920 930 940 950
GGAGCAATCATAGGACTAATGGTAGGAGGGGTAGTCATAGCAGCGGCTTA
CCTCGTTAGTATCCTGATTACCATCCTCCCCATCAGTATCGTCGCCGAAT
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
                960 970 980 990 1000
TGATGCTGAATTTCGTCATGATTCGGGTTATGAAGTTCATCATCAAAAAT
ACTACGACTTAAAGCAGTACTAAGCCCAATACTTCAAGTAGTAGTTTTTA
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
              1010 1020 1030 1040 1050
TAGTGTTTTTCGCTGAAGATGTTGGTTCTAATAAAGGAGCTATTATAGGT
ATCACAAAAAGCGACTTCTACAACCAAGATTATTTCCTCGATAATATCCA
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
                                                                                          >BglII
                                                                                              |
              1060 1070 1080 1090 1100
TTAATGGTTGGGGGTGTTGTTATTGCTGGTGGCGGTTCGAGATCTATCCA
AATTACCAACCCCCACAACAATAACGACCACCGCCAAGCTCTAGATAGGT
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
              1110 1120 1130 1140 1150
GACCGTCAAGGTGAGCGTGAGCGCCGCCACCCTGGGCCTGGGCTTCATCA
CTGGCAGTTCCACTCGCACTCGCGGCGGTGGGACCCGGACCCGAAGTAGT
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
              1160 1170 1180 1190 1200
TCTTCTGCGTGGGGTTCTTCCGGTGGCGCAAGAGCCACTCCTCCAGCTAC
AGAAGACGCACCCCAAGAAGGCCACCGCGTTCTCGGTGAGGAGGTCGATG
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
                                                                                              >HindIII
                                                                                                    |
            1210 1220 1230 1240 |  1250
ACCCCCCTCTCCGGCTCCACCTATCCCGAGGGGCGCCACTAGAAGCTTTC
TGGGGGGAGAGGCCGAGGTGGATAGGGCTCCCCGCGGTGATCTTCGAAAG
_______534 TO 1196 OF P4U-AB42TRIMERAMP.SEQ_______>
___________61 TO 633 OF AB42-TRIMER.ENDO__________>
___79 TO 723 OF HAB42TRIMER.E3.ENDO.SEQ [SPLIT____>
                                        >NotI
                                            |
            1260 1270 1280 1290 1300
TAGTTCTAGAGCACTGGCGGCCGCGACTCTAGATCATAATCAGCCATACC
ATCAAGATCTCGTGACCGCCGGCGCTGAGATCTAGTATTAGTCGGTATGG
________>
            1310 1320 1330 1340 1350
ACATTTGTAGAGGTTTTACTTGCTTTAAAAAACCTCCCACACCTCCCCCT
TGTAAACATCTCCAAAATGAACGAAATTTTTTGGAGGGTGTGGAGGGGGA
                                            >BsmI >HpaI
                                                |  |
                                          >MfeI >HincII
                                              ||  |
            1360 1370 || 1380 1390 1400
GAACCTGAAACATAAAATGAATGCAATTGTTGTTGTTAACTTGTTTATTG
CTTGGACTTTGTATTTTACTTACGTTAACAACAACAATTGAACAAATAAC
              >PsiI
            |
            1410 1420 1430 1440 1450
CAGCTTATAATGGTTACAAATAAAGCAATAGCATCACAAATTTCACAAAT
GTCGAATATTACCAATGTTTATTTCGTTATCGTAGTGTTTAAAGTGTTTA
                                      >BsmI
                                          |
            1460 1470 1480 1490 1500
AAAGCATTTTTTTCACTGCATTCTAGTTGTGGTTTGTCCAAACTCATCAA
TTTCGTAAAAAAAGTGACGTAAGATCAACACCAAACAGGTTTGAGTAGTT
                                                      >SspI
                                                          |
1510 1520 1530 |  1540 1550
    TGTATCTTAAGGCGTAAATTGTAAGCGTTAATATTTTGTTAAAATTCGCG
    ACATAGAATTCCGCATTTAACATTCGCAATTATAAAACAATTTTAAGCGC
            1560 1570 1580 1590 1600
TTAAATTTTTGTTAAATCAGCTCATTTTTTAACCAATAGGCCGAAATCGG
AATTTAAAAACAATTTAGTCGAGTAAAAAATTGGTTATCCGGCTTTAGCC
                          >PsiI
                              |
            1610 |  1620 1630 1640 1650
CAAAATCCCTTATAAATCAAAAGAATAGACCGAGATAGGGTTGAGTGTTG
GTTTTAGGGAATATTTAGTTTTCTTATCTGGCTCTATCCCAACTCACAAC
            1660 1670 1680 1690 1700
TTCCAGTTTGGAACAAGAGTCCACTATTAAAGAACGTGGACTCCAACGTC
AAGGTCAAACCTTGTTCTCAGGTGATAATTTCTTGCACCTGAGGTTGCAG
            1710 1720 1730 1740 1750
AAAGGGCGAAAAACCGTCTATCAGGGCGATGGCCCACTACGTGAACCATC
TTTCCCGCTTTTTGGCAGATAGTCCCGCTACCGGGTGATGCACTTGGTAG
            1760 1770 1780 1790 1800
ACCCTAATCAAGTTTTTTGGGGTCGAGGTGCCGTAAAGCACTAAATCGGA
TGGGATTAGTTCAAAAAACCCCAGCTCCACGGCATTTCGTGATTTAGCCT
            1810 1820 1830 1840 1850
ACCCTAAAGGGAGCCCCCGATTTAGAGCTTGACGGGGAAAGCCGGCGAAC
TGGGATTTCCCTCGGGGGCTAAATCTCGAACTGCCCCTTTCGGCCGCTTG
            1860 1870 1880 1890 1900
GTGGCGAGAAAGGAAGGGAAGAAAGCGAAAGGAGCGGGCGCTAGGGCGCT
CACCGCTCTTTCCTTCCCTTCTTTCGCTTTCCTCGCCCGCGATCCCGCGA
            1910 1920 1930 1940 1950
GGCAAGTGTAGCGGTCACGCTGCGCGTAACCACCACACCCGCCGCGCTTA
CCGTTCACATCGCCAGTGCGACGCGCATTGGTGGTGTGGGCGGCGCGAAT
            1960 1970 1980 1990 2000
ATGCGCCGCTACAGGGCGCGTCAGGTGGCACTTTTCGGGGAAATGTGCGC
TACGCGGCGATGTCCCGCGCAGTCCACCGTGAAAAGCCCCTTTACACGCG
            2010 2020 2030 2040 2050
GGAACCCCTATTTGTTTATTTTTCTAAATACATTCAAATATGTATCCGCT
CCTTGGGGATAAACAAATAAAAAGATTTATGTAAGTTTATACATAGGCGA
                                                          >SspI
                                                              |
            2060 2070 2080 | 2090 2100
CATGAGACAATAACCCTGATAAATGCTTCAATAATATTGAAAAAGGAAGA
GTACTCTGTTATTGGGACTATTTACGAAGTTATTATAACTTTTTCCTTCT
>Bsu36I >PvuII
                                  |
    |  2110 2120 |  2130 2140 2150
GTCCTGAGGCGGAAAGAACCAGCTGTGGAATGTGTGTCAGTTAGGGTGTG
CAGGACTCCGCCTTTCTTGGTCGACACCTTACACACAGTCAATCCCACAC
          2160 2170 2180 2190 2200
GAAAGTCCCCAGGCTCCCCAGCAGGCAGAAGTATGCAAAGCATGCATCTC
CTTTCAGGGGTCCGAGGGGTCGTCCGTCTTCATACGTTTCGTACGTAGAG
          >SexAI
              |
          2210 |  2220 2230 2240 2250
AATTAGTCAGCAACCAGGTGTGGAAAGTCCCCAGGCTCCCCAGCAGGCAG
TTAATCAGTCGTTGGTCCACACCTTTCAGGGGTCCGAGGGGTCGTCCGTC
          2260 2270 2280 2290 2300
AAGTATGCAAAGCATGCATCTCAATTAGTCAGCAACCATAGTCCCGCCCC
TTCATACGTTTCGTACGTAGAGTTAATCAGTCGTTGGTATCAGGGCGGGG
          2310 2320 2330 2340 2350
TAACTCCGCCCATCCCGCCCCTAACTCCGCCCAGTTCCGCCCATTCTCCG
ATTGAGGCGGGTAGGGCGGGGATTGAGGCGGGTCAAGGCGGGTAAGAGGC
          2360 2370 2380 2390 2400
CCCCATGGCTGACTAATTTTTTTTATTTATGCAGAGGCCGAGGCCGCCTC
GGGGTACCGACTGATTAAAAAAAATAAATACGTCTCCGGCTCCGGCGGAG
                                                                                      >AvrII
                                                                                          |
                                                                                    >StuI
                                                                          || 2410
                2420 2430 2440 ||2450
GGCCTCTGAGCTATTCCAGAAGTAGTGAGGAGGCTTTTTTGGAGGCCTAG
CCGGAGACTCGATAAGGTCTTCATCACTCCTCCGAAAAAACCTCCGGATC
              >ClaI
                  |
              >BspDI
                  |
          2460 |  2470 2480 2490 2500
GCTTTTGCAAAGATCGATCAAGAGACAGGATGAGGATCGTTTCGCATGAT
CGAAAACGTTTCTAGCTAGTTCTCTGTCCTACTCCTAGCAAAGCGTACTA
          2510 2520 2530 2540 2550
TGAACAAGATGGATTGCACGCAGGTTCTCCGGCCGCTTGGGTGGAGAGGC
ACTTGTTCTACCTAACGTGCGTCCAAGAGGCCGGCGAACCCACCTCTCCG
          2560 2570 2580 2590 2600
TATTCGGCTATGACTGGGCACAACAGACAATCGGCTGCTCTGATGCCGCC
ATAAGCCGATACTGACCCGTGTTGTCTGTTAGCCGACGAGACTACGGCGG
          2610 2620 2630 2640 2650
GTGT TCCGGCTGTCAGCGCAGGGGCGCCCGGTTCTTTTTGTCAAGACCGA
CACAAGGCCGACAGTCGCGTCCCCGCGGGCCAAGAAAAACAGTTCTGGCT
          2660 2670 2680 2690 2700
CCTGTCCGGTGCCCTGAATGAACTGCAAGACGAGGCAGCGCGGCTATCGT
GGACAGGCCACGGGACTTACTTGACGTTCTGCTCCGTCGCGCCGATAGCA
                                                              >PvuII
                                                                |
          >MscI >FspI |
              |  |  |
              |2710 2720 |2730 2740 2750
GGCTGGCCACGACGGGCGTTCCTTGCGCAGCTGTGCTCGACGTTGTCACT
CCGACCGGTGCTGCCCGCAAGGAACGCGTCGACACGAGCTGCAACAGTGA
          2760 2770 2780 2790 2800
GAAGCGGGAAGGGACTGGCTGCTATTGGGCGAAGTGCCGGGGCAGGATCT
CTTCGCCCTTCCCTGACCGACGATAACCCGCTTCACGGCCCCGTCCTAGA
          2810 2820 2830 2840 2850
CCTGTCATCTCACCTTGCTCCTGCCGAGAAAGTATCCATCATGGCTGATG
GGACAGTAGAGTGGAACGAGGACGGCTCTTTCATAGGTAGTACCGACTAC
        2860 2870 2880 2890 2900
CAATGCGGCGGCTGCATACGCTTGATCCGGCTACCTGCCCATTCGACCAC
GTTACGCCGCCGACGTATGCGAACTAGGCCGATGGACGGGTAAGCTGGTG
        2910 2920 2930 2940 2950
CAAGCGAAACATCGCATCGAGCGAGCACGTACTCGGATGGAAGCCGGTCT
GTTCGCTTTGTAGCGTAGCTCGCTCGTGCATGAGCCTACCTTCGGCCAGA
        2960 2970 2980 2990 3000
TGTCGATCAGGATGATCTGGACGAAGAGCATCAGGGGCTCGCGCCAGCCG
ACAGCTAGTCCTACTAGACCTGCTTCTCGTAGTCCCCGAGCGCGGTCGGC
        3010 3020 3030 3040 3050
AACTGTTCGCCAGGCTCAAGGCGAGCATGCCCGACGGCGAGGATCTCGTC
TTGACAAGCGGTCCGAGTTCCGCTCGTACGGGCTGCCGCTCCTAGAGCAG
        3060 3070 3080 3090 3100
GTGACCCATGGCGATGCCTGCTTGCCGAATATCATGGTGGAAAATGGCCG
CACTGGGTACCGCTACGGACGAACGGCTTATAGTACCACCTTTTACCGGC
                                                                          >RsrI
                                                                              |
                                                                          >AvaII
                                                                              |
        3110 3120 3130 3140 3150
CTTTTCTGGATTCATCGACTGTGGCCGGCTGGGTGTGGCGGACCGCTATC
GAAAAGACCTAAGTAGCTGACACCGGCCGACCCACACCGCCTGGCGATAG
        3160 3170 3180 3190 3200
AGGACATAGCGTTGGCTACCCGTGATATTGCTGAAGAGCTTGGCGGCGAA
TCCTGTATCGCAACCGATGGGCACTATAACGACTTCTCGAACCGCCGCTT
        3210 3220 3230 3240 3250
TGGGCTGACCGCTTCCTCGTGCTTTACGGTATCGCCGCTCCCGATTCGCA
ACCCGACTGGCGAAGGAGCACGAAATGCCATAGCGGCGAGGGCTAAGCGT
        3260 3270 3280 3290 3300
GCGCATCGCCTTCTATCGCCTTCTTGACGAGTTCTTCTGAGCGGGACTCT
CGCGTAGCGGAAGATAGCGGAAGAACTGCTCAAGAAGACTCGCCCTGAGA
  >BstBI
|
        |3310 3320 3330 3340 3350
GGGGTTCGAAATGACCGACCAAGCGACGCCCAACCTGCCATCACGAGATT
CCCCAAGCTTTACTGGCTGGTTCGCTGCGGGTTGGACGGTAGTGCTCTAA
          3360 3370 3380 3390 3400
TCGATTCCACCGCCGCCTTCTATGAAAGGTTGGGCTTCGGAATCGTTTTC
AGCTAAGGTGGCGGCGGAAGATACTTTCCAACCCGAAGCCTTAGCAAAAG
          3410 3420 3430 3440 3450
CGGGACGCCGGCTGGATGATCCTCCAGCGCGGGGATCTCATGCTGGAGTT
GCCCTGCGGCCGACCTACTAGGAGGTCGCGCCCCTAGAGTACGACCTCAA
        >AvrII
            |
          3460|  3470 3480 3490 3500
CTTCGCCCACCCTAGGGGGAGGCTAACTGAAACACGGAAGGAGACAATAC
GAAGCGGGTGGGATCCCCCTCCGATTGACTTTGTGCCTTCCTCTGTTATG
          3510 3520 3530 3540 3550
CGGAAGGAACCCGCGCTATGACGGCAATAAAAAGACAGAATAAAACGCAC
GCCTTCCTTGGGCGCGATACTGCCGTTATTTTTCTGTCTTATTTTGCGTG
                                                          >AvaII
                                                              |
          3560 3570 3580 | 3590 3600
GGTGTTGGGTCGTTTGTTCATAAACGCGGGGTTCGGTCCCAGGGCTGGCA
CCACAACCCAGCAAACAAGTATTTGCGCCCCAAGCCAGGGTCCCGACCGT
        3610 3620 3630 3640 3650
CTCTGTCGATACCCCACCGAGACCCCATTGGGGCCAATACGCCCGCGTTT
GAGACAGCTATGGGGTGGCTCTGGGGTAACCCCGGTTATGCGGGCGCAAA
        3660 3670 3680 3690 3700
CTTCCTTTTCCCCACCCCACCCCCCAAGTTCGGGTGAAGGCCCAGGGCTC
GAAGGAAAAGGGGTGGGGTGGGGGGTTCAAGCCCACTTCCGGGTCCCGAG
                          >EcoO109I >Bsu36I
                                  |  |
        3710 3720 |  3730 |3740 3750
GCAGCCAACGTCGGGGCGGCAGGCCCTGCCATAGCCTCAGGTTACTCATA
CGTCGGTTGCAGCCCCGCCGTCCGGGACGGTATCGGAGTCCAATGAGTAT
        3760 3770 3780 3790 3800
TATACTTTAGATTGATTTAAAACTTCATTTTTAATTTAAAAGGATCTAGG
ATATGAAATCTAACTAAATTTTGAAGTAAAAATTAAATTTTCCTAGATCC
        3810 3820 3830 3840 3850
TGAAGATCCTTTTTGATAATCTCATGACCAAAATCCCTTAACGTGAGTTT
ACTTCTAGGAAAAACTATTAGAGTACTGGTTTTAGGGAATTGCACTCAAA
        3860 3870 3880 3890 3900
TCGTTCCACTGAGCGTCAGACCCCGTAGAAAAGATCAAAGGATCTTCTTG
AGCAAGGTGACTCGCAGTCTGGGGCATCTTTTCTAGTTTCCTAGAAGAAC
        3910 3920 3930 3940 3950
AGATCCTTTTTTTCTGCGCGTAATCTGCTGCTTGCAAACAAAAAAACCAC
TCTAGGAAAAAAAGACGCGCATTAGACGACGAACGTTTGTTTTTTTGGTG
        3960 3970 3980 3990 4000
CGCTACCAGCGGTGGTTTGTTTGCCGGATCAAGAGCTACCAACTCTTTTT
GCGATGGTCGCCACCAAACAAACGGCCTAGTTCTCGATGGTTGAGAAAAA
        4010 4020 4030 4040 4050
CCGAAGGTAACTGGCTTCAGCAGAGCGCAGATACCAAATACTGTCCTTCT
GGCTTCCATTGACCGAAGTCGTCTCGCGTCTATGGTTTATGACAGGAAGA
        4060 4070 4080 4090 4100
AGTGTAGCCGTAGTTAGGCCACCACTTCAAGAACTCTGTAGCACCGCCTA
TCACATCGGCATCAATCCGGTGGTGAAGTTCTTGAGACATCGTGGCGGAT
        4110 4120 4130 4140 4150
CATACCTCGCTCTGCTAATCCTGTTACCAGTGGCTGCTGCCAGTGGCGAT
GTATGGAGCGAGACGATTAGGACAATGGTCACCGACGACGGTCACCGCTA
        4160 4170 4180 4190 4200
AAGTCGTGTCTTACCGGGTTGGACTCAAGACGATAGTTACCGGATAAGGC
TTCAGCACAGAATGGCCCAACCTGAGTTCTGCTATCAATGGCCTATTCCG
        4210 4220 4230 4240 4250
GCAGCGGTCGGGCTGAACGGGGGGTTCGTGCACACAGCCCAGCTTGGAGC
CGTCGCCAGCCCGACTTGCCCCCCAAGCACGTGTGTCGGGTCGAACCTCG
        4260 4270 4280 4290 4300
GAACGACCTACACCGAACTGAGATACCTACAGCGTGAGCTATGAGAAAGC
CTTGCTGGATGTGGCTTGACTCTATGGATGTCGCACTCGATACTCTTTCG
        4310 4320 4330 4340 4350
GCCACGCTTCCCGAAGGGAGAAAGGCGGACAGGTATCCGGTAAGCGGCAG
CGGTGCGAAGGGCTTCCCTCTTTCCGCCTGTCCATAGGCCATTCGCCGTC
        4360 4370 4380 4390 4400
GGTCGGAACAGGAGAGCGCACGAGGGAGCTTCCAGGGGGAAACGCCTGGT
CCAGCCTTGTCCTCTCGCGTGCTCCCTCGAAGGTCCCCCTTTGCGGACCA
        4410 4420 4430 4440 4450
ATCTTTATAGTCCTGTCGGGTTTCGCCACCTCTGACTTGAGCGTCGATTT
TAGAAATATCAGGACAGCCCAAAGCGGTGGAGACTGAACTCGCAGCTAAA
        4460 4470 4480 4490 4500
TTGTGATGCTCGTCAGGGGGGCGGAGCCTATGGAAAAACGCCAGCAACGC
AACACTACGAGCAGTCCCCCCGCCTCGGATACCTTTTTGCGGTCGTTGCG
                                                                                >PciI
                                                                                    |
        4510 4520 4530 4540 |  4550
GGCCTTTTTACGGTTCCTGGCCTTTTGCTGGCCTTTTGCTCACATGTTCT
CCGGAAAAATGCCAAGGACCGGAAAACGACCGGAAAACGAGTGTACAAGA
        4560 4570 4580 4590 4600
TTCCTGCGTTATCCCCTGATTCTGTGGATAACCGTATTACCGCCATGCAT
AAGGACGCAATAGGGGACTAAGACACCTATTGGCATAATGGCGGTACGTA
(SED ID NO: 1)
Pcmv-Gal4.
      >NruI >MluI >SpeI
          |  |  | 10
                                  20 30 40 50
GACTCTTCGCGATGTACGGGCCAGATATACGCGTTGACATTGATTATTGA
CTGAGAAGCGCTACATGCCCGGTCTATATGCGCAACTGTAACTAATAACT
    >AseI
    |
            |60 70 80 90 100
CTAGTTATTAATAGTAATCAATTACGGGGTCATTAGTTCATAGCCCATAT
GATCAATAATTATCATTAGTTAATGCCCCAGTAATCAAGTATCGGGTATA
            110 120 130 140 150
ATGGAGTTCCGCGTTACATAACTTACGGTAAATGGCCCGCCTGGCTGACC
TACCTCAAGGCGCAATGTATTGAATGCCATTTACCGGGCGGACCGACTGG
            160 170 180 190 200
GCCCAACGACCCCCGCCCATTGACGTCAATAATGACGTATGTTCCCATAG
CGGGTTGCTGGGGGCGGGTAACTGCAGTTATTACTGCATACAAGGGTATC
            210 220 230 240 250
TAACGCCAATAGGGACTTTCCATTGACGTCAATGGGTGGACTATTTACGG
ATTGCGGTTATCCCTGAAAGGTAACTGCAGTTACCCACCTGATAAATGCC
                                                            >NdeI
                                                                |
            260 270 280 |  290 300
TAAACTGCCCACTTGGCAGTACATCAAGTGTATCATATGCCAAGTACGCC
ATTTGACGGGTGAACCGTCATGTAGTTCACATAGTATACGGTTCATGCGG
            310 320 330 340 350
CCCTATTGACGTCAATGACGGTAAATGGCCCGCCTGGCATTATGCCCAGT
GGGATAACTGCAGTTACTGCCATTTACCGGGCGGACCGTAATACGGGTCA
                                                                            >SnaBI
                                                                                |
            360 370 380 390|  400
ACATGACCTTATGGGACTTTCCTACTTGGCAGTACATCTACGTATTAGTC
TGTACTGGAATACCCTGAAAGGATGAACCGTCATGTAGATGCATAATCAG
          >NcoI
              |
          >BtgI
              |
            410|  420 430 440 450
ATCGCTATTACCATGGTGATGCGGTTTTGGCAGTACATCAATGGGCGTGG
TAGCGATAATGGTACCACTACGCCAAAACCGTCATGTAGTTACCCGCACC
            460 470 480 490 500
ATAGCGGTTTGACTCACGGGGATTTCCAAGTCTCCACCCCATTGACGTCA
TATCGCCAAACTGAGTGCCCCTAAAGGTTCAGAGGTGGGGTAACTGCAGT
            510 520 530 540 550
ATGGGAGTTTGTTTTGGCACCAAAATCAACGGGACTTTCCAAAATGTCGT
TACCCTCAAACAAAACCGTGGTTTTAGTTGCCCTGAAAGGTTTTACAGCA
            560 570 580 590 600
AACAACTCCGCCCCATTGACGCAAATGGGCGGTAGGCGTGTACGGTGGGA
TTGTTGAGGCGGGGTAACTGCGTTTACCCGCCATCCGCACATGCCACCCT
                        >SacI
                            |
            610 620 630 640 650
GGTCTATATAAGCAGAGCTCTCTGGCTAACTAGAGAACCCACTGCTTACT
CCAGATATATTCGTCTCGAGAGACCGATTGATCTCTTGGGTGACGAATGA
                                                                                                            >BmtI
                                                                                                        |
                >AseI >NheI |
                    |  |  |
            660 |  670 680 690 | 700
GGCTTATCGAAATTAATACGACTCACTATAGGGAGACCCAAGCTGGCTAG
CCGAATAGCTTTAATTATGCTGAGTGATATCCCTCTGGGTTCGACCGATC
                                            >KpnI
                                              |
              >AflII >Acc65I|  >BamHI
                    |  |  |  |
      >PmeI |>HindIII|  |  >SacI >SpeI
            |  |  |  |  |  | |  |
            |  710 |  720 |  730 | 740 750
CGTTTAAACTTAAGCTTGGTACCGAGCTCGGATCCACTAGTCCAGTGTGG
GCAAATTTGAATTCGAACCATGGCTCGAGCCTAGGTGATCAGGTCACACC
>EcoRI
    |
    |  760 770 780 790 800
TGGAATTCCACGCCGCCACCATGAAGCTACTGTCTTCTATCGAACAAGCA
ACCTTAAGGTGCGGCGGTGGTACTTCGATGACAGAAGATAGCTTGTTCGT
                __________1 TO 2658 OF GAL4.DNA___________>
              810 820 830 840 850
TGCGATATTTGCCGACTTAAAAAGCTCAAGTGCTCCAAAGAAAAACCGAA
ACGCTATAAACGGCTGAATTTTTCGAGTTCACGAGGTTTCTTTTTGGCTT
_______1 TO 2658 OF GAL4.DNA______________________>
              860 870 880 890 900
GTGCGCCAAGTGTCTGAAGAACAACTGGGAGTGTCGCTACTCTCCCAAAA
CACGCGGTTCACAGACTTCTTGTTGACCCTCACAGCGATGAGAGGGTTTT
_______1 TO 2658 OF GAL4.DNA______________________>
              910 920 930 940 950
CCAAAAGGTCTCCGCTGACTAGGGCACATCTGACAGAAGTGGAATCAAGG
GGTTTTCCAGAGGCGACTGATCCCGTGTAGACTGTCTTCACCTTAGTTCC
______________1 TO 2658 OF GAL4.DNA_______________>
                                                                              >AvaI
                                                                                          |
                                                                              >BsoBI
                                                                                          |
                                                                            >PaeR7I
                                                                                          |
                                                                              >TliI
                                                                                          |
                                                                              >XhoI
                                                                                          |
                    960 970 980 990 1000
CTAGAAAGACTGGAACAGCTATTTCTACTGATTTTTCCTCGAGAAGACCT
GATCTTTCTGACCTTGTCGATAAAGATGACTAAAAAGGAGCTCTTCTGGA
______________1 TO 2658 OF GAL4.DNA_______________>
                                                                                                          >HpaI
                                                                                                | 1010
                                1020 1030 1040 1050
TGACATGATTTTGAAAATGGATTCTTTACAGGATATAAAAGCATTGTTAA
ACTGTACTAAAACTTTTACCTAAGAAATGTCCTATATTTTCGTAACAATT
______________1 TO 2658 OF GAL4.DNA_______________>
                >BsrGI
                            |
              1060|  1070 1080 1090 1100
CAGGATTATTTGTACAAGATAATGTGAATAAAGATGCCGTCACAGATAGA
GTCCTAATAAACATGTTCTATTACACTTATTTCTACGGCAGTGTCTATCT
______1 TO 2658 OF GAL4.DNA_______________________>
              1110 1120 1130 1140 1150
TTGGCTTCAGTGGAGACTGATATGCCTCTAACATTGAGACAGCATAGAAT
AACCGAAGTCACCTCTGACTATACGGAGATTGTAACTCTGTCGTATCTTA
______1 TO 2658 OF GAL4.DNA_______________________>
              1160 1170 1180 1190 1200
AAGTGCGACATCATCATCGGAAGAGAGTAGTAACAAAGGTCAAAGACAGT
TTCACGCTGTAGTAGTAGCCTTCTCTCATCATTGTTTCCAGTTTCTGTCA
___________1 TO 2658 OF GAL4.DNA__________________>
            >ClaI
                        |
            >BspDI
                        |
            1210 1220 1230 1240 1250
TGACTGTATCGATTGACTCGGCAGCTCATCATGATAACTCCACAATTCCG
ACTGACATAGCTAACTGAGCCGTCGAGTAGTACTATTGAGGTGTTAAGGC
______1 TO 2658 OF GAL4.DNA_______________________>
            1260 1270 1280 1290 1300
TTGGATTTTATGCCCAGGGATGCTCTTCATGGATTTGATTGGTCTGAAGA
AACCTAAAATACGGGTCCCTACGAGAAGTACCTAAACTAACCAGACTTCT
______________1 TO 2658 OF GAL4.DNA_______________>
      >PciI
          |
                |1310 1320 1330 1340 1350
GGATGACATGTCGGATGGCTTGCCCTTCCTGAAAACGGACCCCAACAATA
CCTACTGTACAGCCTACCGAACGGGAAGGACTTTTGCCTGGGGTTGTTAT
______1 TO 2658 OF GAL4.DNA_______________________>
                  1360 1370 1380 1390 1400
ATGGGTTCTTTGGCGACGGTTCTCTCTTATGTATTCTTCGATCTATTGGC
TACCCAAGAAACCGCTGCCAAGAGAGAATACATAAGAAGCTAGATAACCG
______________1 TO 2658 OF GAL4.DNA_______________>
                                                                    >HpaI
                                                                        |
                                                            >AclI |
                                                                |  |
                  1410 1420 1430 |  1440 1450
TTTAAACCGGAAAATTACACGAACTCTAACGTTAACAGGCTCCCGACCAT
AAATTTGGCCTTTTAATGTGCTTGAGATTGCAATTGTCCGAGGGCTGGTA
______________1 TO 2658 OF GAL4.DNA >
                                                      >XbaI
                                                          |
                  1460 1470 |1480 1490 1500
GATTACGGATAGATACACGTTGGCTTCTAGATCCACAACATCCCGTTTAC
CTAATGCCTATCTATGTGCAACCGAAGATCTAGGTGTTGTAGGGCAAATG
______________1 TO 2658 OF GAL4.DNA >
                                                                                              >ApaLI
                                                                                                  |
                  1510 1520 1530 1540 |  1550
TTCAAAGTTATCTCAATAATTTTCACCCCTACTGCCCTATCGTGCACTCA
AAGTTTCAATAGAGTTATTAAAAGTGGGGATGACGGGATAGCACGTGAGT
______1 TO 2658 OF GAL4.DNA_______________________>
                  1560 1570 1580 1590 1600
CCGACGCTAATGATGTTGTATAATAACCAGATTGAAATCGCGTCGAAGGA
GGCTGCGATTACTACAACATATTATTGGTCTAACTTTAGCGCAGCTTCCT
______1 TO 2658 OF GAL4.DNA_______________________>
                  1610 1620 1630 1640 1650
TCAATGGCAAATCCTTTTTAACTGCATATTAGCCATTGGAGCCTGGTGTA
AGTTACCGTTTAGGAAAAATTGACGTATAATCGGTAACCTCGGACCACAT
______1 TO 2658 OF GAL4.DNA_______________________>
                  1660 1670 1680 1690 1700
TAGAGGGGGAATCTACTGATATAGATGTTTTTTACTATCAAAATGCTAAA
ATCTCCCCCTTAGATGACTATATCTACAAAAAATGATAGTTTTACGATTT
______1 TO 2658 OF GAL4.DNA_______________________>
                  1710 1720 1730 1740 1750
TCTCATTTGACGAGCAAGGTCTTCGAGTCAGGTTCCATAATTTTGGTGAC
AGAGTAAACTGCTCGTTCCAGAAGCTCAGTCCAAGGTATTAAAACCACTG
______________1 TO 2658 OF GAL4.DNA_______________>
                                        >NruI
                                            |
            1760 1770 1780 1790 1800
AGCCCTACATCTTCTGTCGCGATATACACAGTGGAGGCAGAAAACAAATA
TCGGGATGTAGAAGACAGCGCTATATGTGTCACCTCCGTCTTTTGTTTAT
______1 TO 2658 OF GAL4.DNA_______________________>
            1810 1820 1830 1840 1850
CTAGCTATAATTTTCACAGCTTTTCCATAAGAATGGCCATATCATTGGGC
GATCGATATTAAAAGTGTCGAAAAGGTATTCTTACCGGTATAGTAACCCG
_______________1 TO 2658 OF GAL4.DNA______________>
              >PpuMI >BsmI
                          |  |
            1860 1870 1880 1890 1900
TTGAATAGGGACCTCCCCTCGTCCTTCAGTGATAGCAGCATTCTGGAACA
AACTTATCCCTGGAGGGGAGCAGGAAGTCACTATCGTCGTAAGACCTTGT
______________1 TO 2658 OF GAL4.DNA_______________>
                                                  >AccI >MfeI
                                                      |  |
            1910 1920 |  1930 1940|  1950
AAGACGCCGAATTTGGTGGTCTGTCTACTCTTGGGAGATCCAATTGTCCC
TTCTGCGGCTTAAACCACCAGACAGATGAGAACCCTCTAGGTTAACAGGG
______1 TO 2658 OF GAL4.DNA_______________________>
            1960 1970 1980 1990 2000
TGCTTTATGGTCGATCCATCCAGCTTTCTCAGAATACAATCTCCTTCCCT
ACGAAATACCAGCTAGGTAGGTCGAAAGAGTCTTATGTTAGAGGAAGGGA
______________1 TO 2658 OF GAL4.DNA_______________>
                >AccI
                    |
              >SalI >PpuMI
            ||  |
            2010 2020 2030 |  2040 2050
TCTTCTGTCGACGATGTGCAGCGTACCACAACAGGTCCCACCATATATCA
AGAAGACAGCTGCTACACGTCGCATGGTGTTGTCCAGGGTGGTATATAGT
______1 TO 2658 OF GAL4.DNA_______________________>
            2060 2070 2080 2090 2100
TGGCATCATTGAAACAGCAAGGCTCTTACAAGTTTTCACAAAAATCTATG
ACCGTAGTAACTTTGTCGTTCCGAGAATGTTCAAAAGTGTTTTTAGATAC
______________1 TO 2658 OF GAL4.DNA_______________>
                                              >PstI
                                                  |
            2110 2120 |  2130 2140 2150
AACTAGACAAAACAGTAACTGCAGAAAAAAGTCCTATATGTGCAAAAAAA
TTGATCTGTTTTGTCATTGACGTCTTTTTTCAGGATATACACGTTTTTTT
______1 TO 2658 OF GAL4.DNA_______________________>
            2160 2170 2180 2190 2200
TGCTTGATGATTTGTAATGAGATTGAGGAGGTTTCGAGACAGGCACCAAA
ACGAACTACTAAACATTACTCTAACTCCTCCAAAGCTCTGTCCGTGGTTT
______1 TO 2658 OF GAL4.DNA_______________________>
                    2210 2220 2230 2240 2250
GTTTTTACAAATGGATATTTCCACCACCGCTCTAACCAATTTGTTGAAGG
CAAAAATGTTTACCTATAAAGGTGGTGGCGAGATTGGTTAAACAACTTCC
______________1 TO 2658 OF GAL4.DNA_______________>
                                                          >BstBI
                                                              |
                    2260 2270 2280 2290 2300
AACACCCTTGGCTATCCTTTACAAGATTCGAACTGAAGTGGAAACAGTTG
TTGTGGGAACCGATAGGAAATGTTCTAAGCTTGACTTCACCTTTGTCAAC
______1 TO 2658 OF GAL4.DNA_______________________>
                  2310 2320 2330 2340 2350
TCTCTTATCATTTATGTATTAAGAGATTTTTTCACTAATTTTACCCAGAA
AGAGAATAGTAAATACATAATTCTCTAAAAAAGTGATTAAAATGGGTCTT
______1 TO 2658 OF GAL4.DNA_______________________>
                  2360 2370 2380 2390 2400
AAAGTCACAACTAGAACAGGATCAAAATGATCATCAAAGTTATGAAGTTA
TTTCAGTGTTGATCTTGTCCTAGTTTTACTAGTAGTTTCAATACTTCAAT
______1 TO 2658 OF GAL4.DNA_______________________>
                  2410 2420 2430 2440 2450
AACGATGCTCCATCATGTTAAGCGATGCAGCACAAAGAACTGTTATGTCT
TTGCTACGAGGTAGTACAATTCGCTACGTCGTGTTTCTTGACAATACAGA
______1 TO 2658 OF GAL4.DNA_______________________>
                  2460 2470 2480 2490 2500
GTAAGTAGCTATATGGACAATCATAATGTCACCCCATATTTTGCCTGGAA
CATTCATCGATATACCTGTTAGTATTACAGTGGGGTATAAAACGGACCTT
______1 TO 2658 OF GAL4.DNA_______________________>
                  2510 2520 2530 2540 2550
TTGTTCTTATTACTTGTTCAATGCAGTCCTAGTACCCATAAAGACTCTAC
AACAAGAATAATGAACAAGTTACGTCAGGATCATGGGTATTTCTGAGATG
______________1 TO 2658 OF GAL4.DNA_______________>
>BsmI
I
                  2560 2570 |  2580 2590 2600
TCTCAAACTCAAAATCGAATGCTGAGAATAACGAGACCGCACAATTATTA
AGAGTTTGAGTTTTAGCTTACGACTCTTATTGCTCTGGCGTGTTAATAAT
______1 TO 2658 OF GAL4.DNA_______________________>
                  2610 2620 2630 2640 2650
CAACAAATTAACACTGTTCTGATGCTATTAAAAAAACTGGCCACTTTTAA
GTTGTTTAATTGTGACAAGACTACGATAATTTTTTTGACCGGTGAAAATT
______________1 TO 2658 OF GAL4.DNA_______________>
                                                              >ScaI
                                                                  |
            2660 2670 2680 2690 2700
AATCCAGACTTGTGAAAAATACATTCAAGTACTGGAAGAGGTATGTGCGC
TTAGGTCTGAACACTTTTTATGTAAGTTCATGACCTTCTCCATACACGCG
______________1 TO 2658 OF GAL4.DNA_______________>
                                                                                                        >PsiI
                                                                                              | 2710
                                2720 2730 2740 |2750
CGTTTCTGTTATCACAGTGTGCAATCCCATTACCGCATATCAGTTATAAC
GCAAAGACAATAGTGTCACACGTTAGGGTAATGGCGTATAGTCAATATTG
______________1 TO 2658 OF GAL4.DNA_______________>
                                                          >SspI
                                                              |
              2760 2770 2780 2790 2800
AATAGTAATGGTAGCGCCATTAAAAATATTGTCGGTTCTGCAACTATCGC
TTATCATTACCATCGCGGTAATTTTTATAACAGCCAAGACGTTGATAGCG
______________1 TO 2658 OF GAL4.DNA_______________>
                                >BspEI
                                    |
              2810 |2820 2830 2840 2850
CCAATACCCTACTCTTCCGGAGGAAAATGTCAACAATATCAGTGTTAAAT
GGTTATGGGATGAGAAGGCCTCCTTTTACAGTTGTTATAGTCACAATTTA
______1 TO 2658 OF GAL4.DNA_______________________>
              2860 2870 2880 2890 2900
ATGTTTCTCCTGGCTCAGTAGGGCCTTCACCTGTGCCATTGAAATCAGGA
TACAAAGAGGACCGAGTCATCCCGGAAGTGGACACGGTAACTTTAGTCCT
______1 TO 2658 OF GAL4.DNA_______________________>
              2910 2920 2930 2940 2950
GCAAGTTTCAGTGATCTAGTCAAGCTGTTATCTAACCGTCCACCCTCTCG
CGTTCAAAGTCACTAGATCAGTTCGACAATAGATTGGCAGGTGGGAGAGC
______1 TO 2658 OF GAL4.DNA_______________________>
              2960 2970 2980 2990 3000
TAACTCTCCAGTGACAATACCAAGAAGCACACCTTCGCATCGCTCAGTCA
ATTGAGAGGTCACTGTTATGGTTCTTCGTGTGGAAGCGTAGCGAGTCAGT
______________1 TO 2658 OF GAL4.DNA_______________>
                                                    >BstAPI
                                                          |
              3010 3020 |3030 3040 3050
CGCCTTTTCTAGGGCAACAGCAACAGCTGCAATCATTAGTGCCACTGACC
GCGGAAAAGATCCCGTTGTCGTTGTCGACGTTAGTAATCACGGTGACTGG
_________________1 TO 2658 OF GAL4.DNA____________>
                                                                                                    >SspI
                                                                                          |  3060
                                3070 3080 3090 | 3100
CCGTCTGCTTTGTTTGGTGGCGCCAATTTTAATCAAAGTGGGAATATTGC
GGCAGACGAAACAAACCACCGCGGTTAAAATTAGTTTCACCCTTATAACG
___________1 TO 2658 OF GAL4.DNA__________________>
                                                                                            >RsrII
                                                                                                |
            3110 3120 3130 3140|  3150
TGATAGCTCATTGTCCTTCACTTTCACTAACAGTAGCAACGGTCCGAACC
ACTATCGAGTAACAGGAAGTGAAAGTGATTGTCATCGTTGCCAGGCTTGG
___________1 TO 2658 OF GAL4.DNA__________________>
                                                    >AfeI >MfeI
                                                            |  |
                3160 3170 3180 3190 3200
TCATAACAACTCAAACAAATTCTCAAGCGCTTTCACAACCAATTGCCTCC
AGTATTGTTGAGTTTGTTTAAGAGTTCGCGAAAGTGTTGGTTAACGGAGG
___________1 TO 2658 OF GAL4.DNA__________________>
          >AclI
        |
              | 3210 3220 3230 3240 3250
TCTAACGTTCATGATAACTTCATGAATAATGAAATCACGGCTAGTAAAAT
AGATTGCAAGTACTATTGAAGTACTTATTACTTTAGTGCCGATCATTTTA
___________1 TO 2658 OF GAL4.DNA__________________>
                                                                >SexAI
                                                                    |
                  3260 3270 3280|  3290 3300
TGATGATGGTAATAATTCAAAACCACTGTCACCTGGTTGGACGGACCAAA
ACTACTACCATTATTAAGTTTTGGTGACAGTGGACCAACCTGCCTGGTTT
___________1 TO 2658 OF GAL4.DNA__________________>
                    >MluI
                        |
                  3310 3320 3330 3340 3350
CTGCGTATAACGCGTTTGGAATCACTACAGGGATGTTTAATACCACTACA
GACGCATATTGCGCAAACCTTAGTGATGTCCCTACAAATTATGGTGATGT
______1 TO 2658 OF GAL4.DNA_______________________>
                  3360 3370 3380 3390 3400
ATGGATGATGTATATAACTATCTATTCGATGATGAAGATACCCCACCAAA
TACCTACTACATATATTGATAGATAAGCTACTACTTCTATGGGGTGGTTT
____________1 TO 2658 OF GAL4.DNA_________________>
                                  >AvaI
                                    |
                                >BsoBI
                                    |
                              >PaeR7I
                                    |
                            >PspXI >XbaI
                                    |  |
                  >NotI >Til|  |  >ApaI
                        |  |  |  |
                  >EagI >XhoI | >PspOMI|  >PmeI
                  |  |  |  |  |  |  3410
                3420 |  3430 |3440 | 3450
CCCAAAAAAAGAGTAAGCGGCCGCTCGAGTCTAGAGGGCCCGTTTAAACC
GGGTTTTTTTCTCATTCGCCGGCGAGCTCAGATCTCCCGGGCAAATTTGG
___1 TO 2658____>
          3460 3470 3480 3490 3500
CGCTGATCAGCCTCGACTGTGCCTTCTAGTTGCCAGCCATCTGTTGTTTG
GCGACTAGTCGGAGCTGACACGGAAGATCAACGGTCGGTAGACAACAAAC
          3510 3520 3530 3540 3550
CCCCTCCCCCGTGCCTTCCTTGACCCTGGAAGGTGCCACTCCCACTGTCC
GGGGAGGGGGCACGGAAGGAACTGGGACCTTCCACGGTGAGGGTGACAGG
          3560 3570 3580 3590 3600
TTTCCTAATAAAATGAGGAAATTGCATCGCATTGTCTGAGTAGGTGTCAT
AAAGGATTATTTTACTCCTTTAACGTAGCGTAACAGACTCATCCACAGTA
          3610 3620 3630 3640 3650
TCTATTCTGGGGGGTGGGGTGGGGCAGGACAGCAAGGGGGAGGATTGGGA
AGATAAGACCCCCCACCCCACCCCGTCCTGTCGTTCCCCCTCCTAACCCT
          3660 3670 3680 3690 3700
AGACAATAGCAGGCATGCTGGGGATGCGGTGGGCTCTATGGCTTCTACTG
TCTGTTATCGTCCGTACGACCCCTACGCCACCCGAGATACCGAAGATGAC
          3710 3720 3730 3740 3750
GGCGGTTTTATGGACAGCAAGCGAACCGGAATTGCCAGCTGGGGCGCCCT
CCGCCAAAATACCTGTCGTTCGCTTGGCCTTAACGGTCGACCCCGCGGGA
          3760 3770 3780 3790 3800
CTGGTAAGGTTGGGAAGCCCTGCAAAGTAAACTGGATGGCTTTCTCGCCG
GACCATTCCAACCCTTCGGGACGTTTCATTTGACCTACCGAAAGAGCGGC
          3810 3820 3830 3840 3850
CCAAGGATCTGATGGCGCAGGGGATCAAGCTCTGATCAAGAGACAGGATG
GGTTCCTAGACTACCGCGTCCCCTAGTTCGAGACTAGTTCTCTGTCCTAC
                                                                                      >EagI
                                                                                | 3860
                3870 3880 3890 3900
AGGATCGTTTCGCATGATTGAACAAGATGGATTGCACGCAGGTTCTCCGG
TCCTAGCAAAGCGTACTAACTTGTTCTACCTAACGTGCGTCCAAGAGGCC
          3910 3920 3930 3940 3950
CCGCTTGGGTGGAGAGGCTATTCGGCTATGACTGGGCACAACAGACAATC
GGCGAACCCACCTCTCCGATAAGCCGATACTGACCCGTGTTGTCTGTTAG
          3960 3970 3980 3990 4000
GGCTGCTCTGATGCCGCCGTGTTCCGGCTGTCAGCGCAGGGGCGCCCGGT
CCGACGAGACTACGGCGGCACAAGGCCGACAGTCGCGTCCCCGCGGGCCA
          4010 4020 4030 4040 4050
TCTTTTTGTCAAGACCGACCTGTCCGGTGCCCTGAATGAACTGCAAGACG
AGAAAAACAGTTCTGGCTGGACAGGCCACGGGACTTACTTGACGTTCTGC
                                                                                  >FspI
                                                                                      |
          4060 4070 4080 4090 |  4100
AGGCAGCGCGGCTATCGTGGCTGGCCACGACGGGCGTTCCTTGCGCAGCT
TCCGTCGCGCCGATAGCACCGACCGGTGCTGCCCGCAAGGAACGCGTCGA
          4110 4120 4130 4140 4150
GTGCTCGACGTTGTCACTGAAGCGGGAAGGGACTGGCTGCTATTGGGCGA
CACGAGCTGCAACAGTGACTTCGCCCTTCCCTGACCGACGATAACCCGCT
          4160 4170 4180 4190 4200
AGTGCCGGGGCAGGATCTCCTGTCATCTCACCTTGCTCCTGCCGAGAAAG
TCACGGCCCCGTCCTAGAGGACAGTAGAGTGGAACGAGGACGGCTCTTTC
          4210 4220 4230 424 4250
TATCCATCATGGCTGATGCAATGCGGCGGCTGCATACGCTTGATCCGGCT
ATAGGTAGTACCGACTACGTTACGCCGCCGACGTATGCGAACTAGGCCGA
          4260 4270 4280 4290 4300
ACCTGCCCATTCGACCACCAAGCGAAACATCGCATCGAGCGAGCACGTAC
TGGACGGGTAAGCTGGTGGTTCGCTTTGTAGCGTAGCTCGCTCGTGCATG
        >BsrFI
            |
          4310|  4320 4330 4340 4350
TCGGATGGAAGCCGGTCTTGTCGATCAGGATGATCTGGACGAAGAGCATC
AGCCTACCTTCGGCCAGAACAGCTAGTCCTACTAGACCTGCTTCTCGTAG
          4360 4370 4380 4390 4400
AGGGGCTCGCGCCAGCCGAACTGTTCGCCAGGCTCAAGGCGAGCATGCCC
TCCCCGAGCGCGGTCGGCTTGACAAGCGGTCCGAGTTCCGCTCGTACGGG
                                  >BtgI
                                      |
                                  >NcoI
                                      |
          4410 4420 |  4430 4440 4450
GACGGCGAGGATCTCGTCGTGACCCATGGCGATGCCTGCTTGCCGAATAT
CTGCCGCTCCTAGAGCAGCACTGGGTACCGCTACGGACGAACGGCTTATA
                                                                                  >NaeI
                                                                                      |
                                                                            >NgoMIV1
                                                                                  | |
                                                                              >BsrFI
                                                                                  | |
          4460 4470 4480 4490 | |  4500
CATGGTGGAAAATGGCCGCTTTTCTGGATTCATCGACTGTGGCCGGCTGG
GTACCACCTTTTACCGGCGAAAAGACCTAAGTAGCTGACACCGGCCGACC
  >RsrII
      |
          4510 4520 4530 4540 4550
GTGTGGCGGACCGCTATCAGGACATAGCGTTGGCTACCCGTGATATTGCT
CACACCGCCTGGCGATAGTCCTGTATCGCAACCGATGGGCACTATAACGA
                                                        >BssSI
                                                            |
          4560 4570 4580 |  4590 4600
GAAGAGCTTGGCGGCGAATGGGCTGACCGCTTCCTCGTGCTTTACGGTAT
CTTCTCGAACCGCCGCTTACCCGACTGGCGAAGGAGCACGAAATGCCATA
          4610 4620 4630 4640 4650
CGCCGCTCCCGATTCGCAGCGCATCGCCTTCTATCGCCTTCTTGACGAGT
GCGGCGAGGGCTAAGCGTCGCGTAGCGGAAGATAGCGGAAGAACTGCTCA
          4660 4670 4680 4690 4700
TCTTCTGAATTATTAACGCTTACAATTTCCTGATGCGGTATTTTCTCCTT
AGAAGACTTAATAATTGCGAATGTTAAAGGACTACGCCATAAAAGAGGAA
          4710 4720 4730 4740 4750
ACGCATCTGTGCGGTATTTCACACCGCATACAGGTGGCACTTTTCGGGGA
TGCGTAGACACGCCATAAAGTGTGGCGTATGTCCACCGTGAAAAGCCCCT
          4760 4770 4780 4790 4800
AATGTGCGCGGAACCCCTATTTGTTTATTTTTCTAAATACATTCAAATAT
TTACACGCGCCTTGGGGATAAACAAATAAAAAGATTTATGTAAGTTTATA
                                                                                            >PmlI
                                                                                  | 4810
                4820 4830 4840 4850
GTATCCGCTCATGAGACAATAACCCTGATAAATGCTTCAATAATAGCACG
CATAGGCGAGTACTCTGTTATTGGGACTATTTACGAAGTTATTATCGTGC
          4860 4870 4880 4890 4900
TGCTAAAACTTCATTTTTAATTTAAAAGGATCTAGGTGAAGATCCTTTTT
ACGATTTTGAAGTAAAAATTAAATTTTCCTAGATCCACTTCTAGGAAAAA
          4910 4920 4930 4940 4950
GATAATCTCATGACCAAAATCCCTTAACGTGAGTTTTCGTTCCACTGAGC
CTATTAGAGTACTGGTTTTAGGGAATTGCACTCAAAAGCAAGGTGACTCG
          4960 4970 4980 4990 5000
GTCAGACCCCGTAGAAAAGATCAAAGGATCTTCTTGAGATCCTTTTTTTC
CAGTCTGGGGCATCTTTTCTAGTTTCCTAGAAGAACTCTAGGAAAAAAAG
          5010 5020 5030 5040 5050
TGCGCGTAATCTGCTGCTTGCAAACAAAAAAACCACCGCTACCAGCGGTG
ACGCGCATTAGACGACGAACGTTTGTTTTTTTGGTGGCGATGGTCGCCAC
          5060 5070 5080 5090 5100
GTTTGTTTGCCGGATCAAGAGCTACCAACTCTTTTTCCGAAGGTAACTGG
CAAACAAACGGCCTAGTTCTCGATGGTTGAGAAAAAGGCTTCCATTGACC
          5110 5120 5130 5140 5150
CTTCAGCAGAGCGCAGATACCAAATACTGTCCTTCTAGTGTAGCCGTAGT
GAAGTCGTCTCGCGTCTATGGTTTATGACAGGAAGATCACATCGGCATCA
          5160 5170 5180 5190 5200
TAGGCCACCACTTCAAGAACTCTGTAGCACCGCCTACATACCTCGCTCTG
ATCCGGTGGTGAAGTTCTTGAGACATCGTGGCGGATGTATGGAGCGAGAC
          5210 5220 5230 5240 5250
CTAATCCTGTTACCAGTGGCTGCTGCCAGTGGCGATAAGTCGTGTCTTAC
GATTAGGACAATGGTCACCGACGACGGTCACCGCTATTCAGCACAGAATG
          5260 5270 5280 5290 5300
CGGGTTGGACTCAAGACGATAGTTACCGGATAAGGCGCAGCGGTCGGGCT
GCCCAACCTGAGTTCTGCTATCAATGGCCTATTCCGCGTCGCCAGCCCGA
                                  >ApaLI
                                                |
          5310 |  5320 5330 5340 5350
GAACGGGGGGTTCGTGCACACAGCCCAGCTTGGAGCGAACGACCTACACC
CTTGCCCCCCAAGCACGTGTGTCGGGTCGAACCTCGCTTGCTGGATGTGG
          5360 5370 5380 5390 5400
GAACTGAGATACCTACAGCGTGAGCTATGAGAAAGCGCCACGCTTCCCGA
CTTGACTCTATGGATGTCGCACTCGATACTCTTTCGCGGTGCGAAGGGCT
          5410 5420 5430 5440 5450
AGGGAGAAAGGCGGACAGGTATCCGGTAAGCGGCAGGGTCGGAACAGGAG
TCCCTCTTTCCGCCTGTCCATAGGCCATTCGCCGTCCCAGCCTTGTCCTC
                                          >BssSI
      |
      | 5460 5470 5480 5490 5500
AGCGCACGAGGGAGCTTCCAGGGGGAAACGCCTGGTATCTTTATAGTCCT
TCGCGTGCTCCCTCGAAGGTCCCCCTTTGCGGACCATAGAAATATCAGGA
          5510 5520 5530 5540 5550
GTCGGGTTTCGCCACCTCTGACTTGAGCGTCGATTTTTGTGATGCTCGTC
CAGCCCAAAGCGGTGGAGACTGAACTCGCAGCTAAAAACACTACGAGCAG
          5560 5570 5580 5590 5600
AGGGGGGCGGAGCCTATGGAAAAACGCCAGCAACGCGGCCTTTTTACGGT
TCCCCCCGCCTCGGATACCTTTTTGCGGTCGTTGCGCCGGAAAAATGCCA
                                      >PciI
                                          |
5610 5620 5630
TCCTGGGCTTTTGCTGGCCTTTTGCTCACATGTTCTT
AGGACCCGAAAACGACCGGAAAACGAGTGTACAAGAA
(SEQ ID NO.: 4)
```

```
ABeta 42 Peptide
(SEQ ID NO.: 3)
Asp Ala Glu Phe Arg His Asp Ser Gly Tyr Glu
1 10
Val His His Gln Lys Leu Val Phe Phe Ala Glu
                        15 20
Asp Val Gly Ser Asn Lys Gly Ala Ile Ile Gly
                25 30
Leu Met Val Gly Gly Val Val Ile Ala
        35 40
```

Intra-muscular delivery of aBeta 42 Alzheimer Disease DNA vaccine without the need for gene gun and gold particles.

A major obstacle for commercialization of the AD vaccine has been delivery of the AD vaccine vector under sterile conditions and under practical conditions for patient delivery. The Gene Gun appears problematic with the pharmaceutical industry and the FDA regulatory agency. Other modes of deliver have been addressed by above and by others, such as electroporation, which was been found to be inefficient.

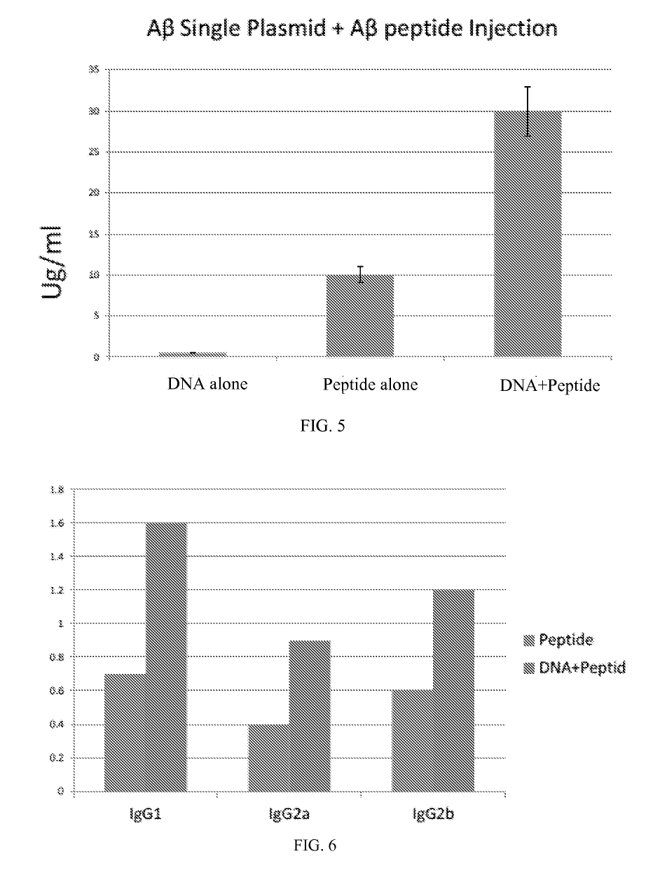

Surprisingly, the inventors have shown herein that intramuscular injection of the AD Single DNA Vector and small concentration of the ABeta 42 peptide elicits very high antibody titer against the ABeta 42 Peptide as indicated in FIG. 5.

FIG. 5 is a graph that shows the results from 4 muscle injections (once a week (20 ug) with trimer DNA+10 ug Aβ peptide (or separate injection) and tested the antibodies at 6 weeks. It was found that DNA+Peptide without adjuvant elicit a better immune response.

The antibody isotype profile was analyzed to determine the balance of the Th1/Th2 response. Surprisingly, it was found that the approach of the present invention did not require an adjuvant. A simple, rapid method of injecting the composition taught herein is greatly enhances clinical trials and clinical use for patients.

Isotyping of antibody generated using DNA and abeta 45 peptide delivered. By intra-muscular injection: the trimer single DNA vector (pv1-h3) 20 ug+10 ug abeta 42 peptide were injected into mouse muscle once per week for a total of four weeks. Serum was obtained from the mouse and tested for abeta isotype antibodies at 6 weeks using an ELISA method.

As can be seen in FIG. 6, trimer DNA+peptide without adjuvant elicited a better immune response compared to peptide alone. Higher isotype antibodies levels were achieved with the DNA+peptide. Both groups induced the Th1 and Th2 reactions but with a predominance of Th2 (IgG1 and IgG2a).

FIG. 6 is a graph that shows the results from 4 muscle injections 4 times (once a week) muscle injection (20 ug Trimer DNA+10 ug Aβ peptide), the serum was tested for Abeta isotype antibodies at 6 weeks with ELISA method. It was found that DNA+Peptide without adjuvant elicited a better immune response compare to peptide alone. Higher isotype antibodies level achieved in DNA+Peptide, but both group induced Th1 and Th2 reaction with predominantly Th2 (IgG1 and IgG2a) bias.

Thus, it was found that the trimer DNA vector can be delivered by intra-muscular injections without the need of the gene gun or gold particles. Furthermore, the levels of antibodies (30 ug/ml) are significantly higher than the DNA (3 ug/ml) or the peptide alone (10 ug/ml) by intramuscular injection or injected intravenously. In addition, the antibody generated was primarily a Th2 response (IgG1 and IgG2a) as indicated in FIG. 5.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A method for diagnosis, treatment or prevention of Alzheimer's Disease comprising:
obtaining a biological sample from a subject suspected of having Alzheimer's Disease;
determining the level of expression of HSP 27, wherein a statistically significant increase in HSP27 protein expression in the sample as compared to a sample from a non-Alzheimer's patient is indicative that the subject has Alzheimer's Disease; and
modifying a treatment of the subject as a result of the detection of Alzheimer's Disease by providing the subject with standard therapy or a composition comprising a single vector that expresses an Aβ42 trimer peptide, wherein the composition triggers an immune response to the Aβ42 trimer peptide, wherein the DNA vector and the Aβ42 trimer peptide are injected intramuscularly, without the need for a gene gun or gold particles, to trigger an immune response to the Aβ42 peptide.

2. (canceled)

3. The method of claim 1, wherein the subject is a human.

4. The method of claim 1, wherein the treatment comprises providing the subject with a vector of SEQ ID NO: 1 and the peptide of SEQ ID NO: 3.

5. The method of claim 1, wherein the level of HSP 27 is determined by measuring protein expression, and the method is selected from fluorescence detection, chemiluminescence detection, electrochemiluminescence detection and patterned arrays, antibody binding, fluorescence activated sorting, detectable bead sorting, antibody arrays, microarrays, enzymatic arrays, receptor binding arrays, solid-phase binding arrays, liquid phase binding arrays, fluorescent resonance transfer, or radioactive labeling.

6. The method of claim 1, wherein the level of expression of HSP27 is determined at the nucleic acid level, and the method is selected from fluorescence detection, chemiluminescence detection, electrochemiluminescence detection and patterned arrays, reverse transcriptase-polymerase chain reaction, detectable bead sorting, microarrays, enzymatic arrays, allele specific primer extension, target specific primer extension, solid-phase binding arrays, liquid phase binding arrays, fluorescent resonance transfer, or radioactive labeling.

7. The method of claim 1, wherein the level of expression of HSP27 is higher than 85, 90, 95, 100, 110, 115, 120, 125, 130, 145, 150, 275, 300, or 315 ng/ml HSP27 in a blood sample.

8. The method of claim 1, wherein the level of expression of HSP27 is higher than 105, 130, 145, 150, 275, 300, or 315 ng/ml HSP27 in a blood sample.

9. The method of claim 1, wherein the expressed Aβ42 trimer peptide triggers a non-inflammatory IgG1 response.

10. The method of claim 1, wherein the Aβ42 trimer peptide and the expressed Aβ42 trimer peptide are effective to trigger an immune response to the Aβ42 trimer peptide without an adjuvant.

11. A method to evaluate a candidate drug believed to be useful in treating Alzheimer's Disease, the method comprising:
(a) measuring the level of expression of HSP27 from a sample obtained from an Alzheimer's Disease patient;
(b) administering a candidate drug to a first subset of the patients, and a placebo to a second subset of the patients, wherein the candidate drug comprises a single DNA vector encoding an Aβ42 trimer peptide; and
(c) determining if the level of expression of HSP27 or the symptoms of Alzheimer's Disease decreased in the first set of patient as compared to the second subset of patients, wherein a statistically significant decrease is indicative that the candidate drug is useful for treating Alzheimer's Disease, wherein the drug candidate further comprises the addition of an Aβ42 peptide, wherein the DNA vector expressing the Aβ42 trimer peptide and the Aβ42 peptide are injected intramuscularly, without the need for a gene gun or gold particles, to trigger an immune response to the Aβ42 peptide.

12. (canceled)

13. The method of claim 11, wherein the subject is a human.

14. The method of claim 11, wherein the DNA vector encodes the Aβ42 trimer peptide is SEQ ID NO: 1, and the Aβ42 peptide is SEQ ID NO: 3.

15. The method of claim 11, wherein the level of HSP 27 is determined by measuring protein expression, and the method is selected from fluorescence detection, chemiluminescence detection, electrochemiluminescence detection and patterned arrays, antibody binding, fluorescence activated sorting, detectable bead sorting, antibody arrays, microarrays, enzymatic arrays, receptor binding arrays, solid-phase binding arrays, liquid phase binding arrays, fluorescent resonance transfer, or radioactive labeling.

16. The method of claim 11, wherein the level of expression of HSP27 is determined at the nucleic acid level, and the method is selected from fluorescence detection, chemiluminescence detection, electrochemiluminescence detection and patterned arrays, reverse transcriptase-polymerase chain reaction, detectable bead sorting, microarrays, enzymatic arrays, allele specific primer extension, target specific primer extension, solid-phase binding arrays, liquid phase binding arrays, fluorescent resonance transfer, or radioactive labeling.

17. The method of claim 11, wherein the level of expression of HSP27 is higher than 85, 90, 95, 100, 110, 115, 120, 125, 130, 145, 150, 275, 300, or 315 ng/ml HSP27 in a blood sample.

18. The method of claim 11, wherein the level of expression of HSP27 is higher than 105, 130, 145, 150, 275, 300, or 315 ng/ml HSP27 in a blood sample.

19. A vector comprising:
a single nucleic acid that comprises in the following order, a viral gene leader sequence, a Aβ42 trimer sequence, and an endosomal targeting sequence, wherein the vector is PV1-H3 and is adapted for is used to treat or prevent Alzheimer's Disease.

20. The vector of claim 19, wherein the viral gene leader sequence is an adenovirus E3 gene leader sequence.

21. The vector of claim 19, further comprises a CMV promoter upstream from the nucleic acid.

22. The vector of claim 19, wherein the vector comprises SEQ ID NO: 1.

23. The vector of claim 19, wherein the endosomal targeting sequence is DXXLL (SEQ ID NO: 2).

24. (canceled)

25. A composition for ameliorating the symptoms of Alzheimer's Disease with a composition comprising an Aβ42 peptide and a DNA vector that expresses an Aβ42 trimer peptide in an amount sufficient to ameliorate the symptoms of Alzheimer's Disease, wherein the composition triggers an immune response to the Aβ42 peptide, wherein the vector comprises SEQ ID NO: 1.

26. The composition of claim 25, wherein the DNA vector and the Aβ42 peptide and the DNA vector are injected intramuscularly without the need for a gene gun or gold particles.

27. The composition of claim 25, wherein the Aβ42 peptide is provided at a subtoxic dose.

28. The composition of claim 25, wherein the composition is provided without an adjuvant.

29. The composition of claim 25, wherein the composition leads to a predominantly Th2 response.

30. The composition of claim 25, wherein the peptide comprises SEQ ID NO: 3.

31. (canceled)

32. The composition of claim 25, wherein the composition consists essentially of the vector of SEQ ID NO: 1 and the peptide of SEQ ID NO: 3.

33. The composition of claim 25, wherein the DNA vector is a single DNA vector.

34. A composition for ameliorating the symptoms of Alzheimer's Disease comprising both an Aβ42 peptide and a DNA vector that expresses an Aβ42 trimer peptide in an amount sufficient to ameliorate the symptoms of Alzheimer's Disease, wherein the Aβ42 peptide and the DNA vector are injected intramuscularly without the need for a gene gun or gold particles, and wherein the composition triggers an immune response to the Aβ42 peptide, wherein the DNA vector comprises SEQ ID NO:1.

35. The composition of claim 34, wherein the Aβ42 peptide and the DNA vector that expresses the Aβ42 trimer peptide are both provided without an adjuvant.

36. The composition of claim 34, wherein the DNA vector is a single DNA vector.

37. The composition of claim 34, wherein the composition leads to a predominantly Th2 response.

38. The composition of claim 34, wherein the peptide comprises SEQ ID NO: 3.

39. (canceled)

40. The composition of claim 34, wherein the composition consists essentially of the vector of SEQ ID NO: 1 and the peptide of SEQ ID NO: 3.

41. A method for the treatment or prevention of Alzheimer's Disease comprising injecting both an Aβ42 peptide and a DNA vector that expresses an Aβ42 trimer peptide, wherein the Aβ42 peptide and the DNA vector are adapted for injection intramuscularly without the need for a gene gun or gold particles, wherein the composition triggers an immune response to the Aβ42 peptide, wherein the DNA vector comprises SEQ ID NO:1.

42. The method of claim 41, wherein the injection triggers a non-inflammatory IgG1 response.

43. The method of claim 41, wherein the Aβ42 peptide and the DNA vector that expresses the Aβ42 trimer peptide are both provided without an adjuvant.

44. The method of claim 41, wherein the DNA vector is a single DNA vector.

45. The method of claim 41, wherein the composition leads to a predominantly Th2 response.

46. The method of claim 41, wherein the Aβ42 peptide comprises SEQ ID NO: 3.

47. (canceled)

48. The method of claim 41, wherein the composition consists essentially of the vector of SEQ ID NO: 1 and the peptide of SEQ ID NO: 3.


SEQUENCE LISTING (4 sequences)

SEQ ID NO: 1 - DNA, 4600 bp, Homo sapiens
tagttattaattacgtaggcttaactatgcggcatcagagcagattgtactgagagtgcaccataagcttgcatgcctgcaggtcgaagcggagtactgtcctccgagcggagtactgtcctccgagcggagtactgtccttcgagcggagtactgtcctccgagtcgactctagagggtatataatggatctcgagatgtaccgagctcttacgcgtgctagcccgggctcgagatctgggcggtaggcgtgtacggtgggaggtctatataagcagagctcgtttagtgaaccgtcagatcactagaagctagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagaattccacgccgccaccatgggctacatgatcctgggcctcctggccctggcggccgtgtgcagcgctgccacgcgtggaggcgggagcgacgccgagttccgccacgacagcggctacgaggtgcaccaccagaagctggtgttcttcgccgaggacgtgggcagcaacaagggcgccatcatcggcctgatggtgggcggcgtggtgatcgccgcagcctacgatgcggaatttcgacatgacagtggatatgaagtacatcaccaaaaactcgtatttttcgcggaagatgtaggaagcaacaagggagcaatcataggactaatggtaggaggggtagtcatagcagcggcttatgatgctgaatttcgtcatgattcgggttatgaagttcatcatcaaaaattagtgtttttcgctgaagatgttggttctaataaaggagctattataggtttaatggttgggggtgttgttattgctggtggcggttcgagatctatccagaccgtcaaggtgagcgtgagcgccgccaccctgggcctgggcttcatcatcttctgcgtggggttcttccggtggcgcaagagccactcctccagctacacccccctctccggctccacctatcccgaggggcgccactagaagctttctagttctagagcactggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 2 - protein, 5 aa, Artificial Sequence
  Synthetic peptide
DXXLL

SEQ ID NO: 3 - protein, 42 aa, Artificial Sequence [sequence omitted]

SEQ ID NO: 4 - DNA, 5637 bp, Homo sapiens
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattccacgccgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgattgactcggcagctcatcatgataactccacaattccgttggattttatgcccagggatgctcttcatggatttgattggtctgaagaggatgacatgtcggatggcttgcccttcctgaaaacggaccccaacaataatgggttctttggcgacggttctctcttatgtattcttcgatctattggctttaaaccggaaaattacacgaactctaacgttaacaggctcccgaccatgattacggatagatacacgttggcttctagatccacaacatcccgtttacttcaaagttatctcaataattttcacccctactgccctatcgtgcactcaccgacgctaatgatgttgtataataaccagattgaaatcgcgtcgaaggatcaatggcaaatcctttttaactgcatattagccattggagcctggtgtatagagggggaatctactgatatagatgttttttactatcaaaatgctaaatctcatttgacgagcaaggtcttcgagtcaggttccataattttggtgacagccctacatcttctgtcgcgatatacacagtggaggcagaaaacaaatactagctataattttcacagcttttccataagaatggccatatcattgggcttgaatagggacctcccctcgtccttcagtgatagcagcattctggaacaaagacgccgaatttggtggtctgtctactcttgggagatccaattgtccctgctttatggtcgatccatccagctttctcagaatacaatctccttcccttcttctgtcgacgatgtgcagcgtaccacaacaggtcccaccatatatcatggcatcattgaaacagcaaggctcttacaagttttcacaaaaatctatgaactagacaaaacagtaactgcagaaaaaagtcctatatgtgcaaaaaaatgcttgatgatttgtaatgagattgaggaggtttcgagacaggcaccaaagtttttacaaatggatatttccaccaccgctctaaccaatttgttgaaggaacacccttggctatcctttacaagattcgaactgaagtggaaacagttgtctcttatcatttatgtattaagagattttttcactaattttacccagaaaaagtcacaactagaacaggatcaaaatgatcatcaaagttatgaagttaaacgatgctccatcatgttaagcgatgcagcacaaagaactgttatgtctgtaagtagctatatggacaatcataatgtcaccccatattttgcctggaattgttcttattacttgttcaatgcagtcctagtacccataaagactctactctcaaactcaaaatcgaatgctgagaataacgagaccgcacaattattacaacaaattaacactgttctgatgctattaaaaaaactggccacttttaaaatccagacttgtgaaaaatacattcaagtactggaagaggtatgtgcgccgtttctgttatcacagtgtgcaatcccattaccgcatatcagttataacaatagtaatggtagcgccattaaaaatattgtcggttctgcaactatcgcccaataccctactcttccggaggaaaatgtcaacaatatcagtgttaaatatgtttctcctggctcagtagggccttcacctgtgccattgaaatcaggagcaagtttcagtgatctagtcaagctgttatctaaccgtccaccctctcgtaactctccagtgacaataccaagaagcacaccttcgcatcgctcagtcacgccttttctagggcaacagcaacagctgcaatcattagtgccactgaccccgtctgctttgtttggtggcgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt